(19) United States
(12) Patent Application Publication
(10) Pub. No.: US 2026/0253702 A1
(43) Pub. Date: Aug. 27, 2026

(54) SYSTEM

(71) Applicant: SoftBank Group Corp., Tokyo (JP)

(72) Inventor: Yusuke SUGANUMA, Tokyo (JP)

(73) Assignee: SoftBank Group Corp., Tokyo (JP)

(21) Appl. No.: 19/542,698

(22) Filed: Feb. 18, 2026

(30) Foreign Application Priority Data
Feb. 21, 2025 (JP) 2025-027005

(51) Int. Cl. G16H 20/17; A61B 5/00; A61B 5/11; A61B 5/16; A61M 5/172; G06F 40/40; G06V 10/82; G06V 40/16; G10L 25/30; G10L 25/63

(52) U.S. Cl. CPC G16H 20/17; A61B 5/1112; A61B 5/165; A61B 5/7264; A61B 5/747; A61M 5/1723; A61M 2230/201; A61M 2230/63; G06F 40/40; G06V 10/82; G06V 40/174; G10L 25/30; G10L 25/63

(57) ABSTRACT

The system according to the embodiment comprises a collection unit, an analysis unit, and a notification unit. The collection unit collects vital data. The analysis unit analyzes data collected by the collection unit and predicts changes in blood glucose levels. The notification unit notifies the timing of insulin injection based on the changes in blood glucose levels predicted by the analysis unit. The notification unit notifies the user and contacts when hypoglycemia is detected.

### CROSS-REFERENCE TO RELATED APPLICATION

The present application claims priority to and incorporates by reference the entire contents of Japanese Patent Application No. 2025-027005 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, it has been difficult to predict changes in blood glucose levels and perform insulin injections at appropriate timings, and there has been a problem in that means for promptly notifying the risk of hypoglycemia are lacking.

### SUMMARY OF THE INVENTION

The system according to the embodiment comprises a collection unit, an analysis unit, and a notification unit. The collection unit collects vital data. The analysis unit analyzes data collected by the collection unit and predicts changes in blood glucose levels. The notification unit notifies the timing of insulin injection based on the changes in blood glucose levels predicted by the analysis unit. The notification unit notifies the user and contacts when hypoglycemia is detected.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

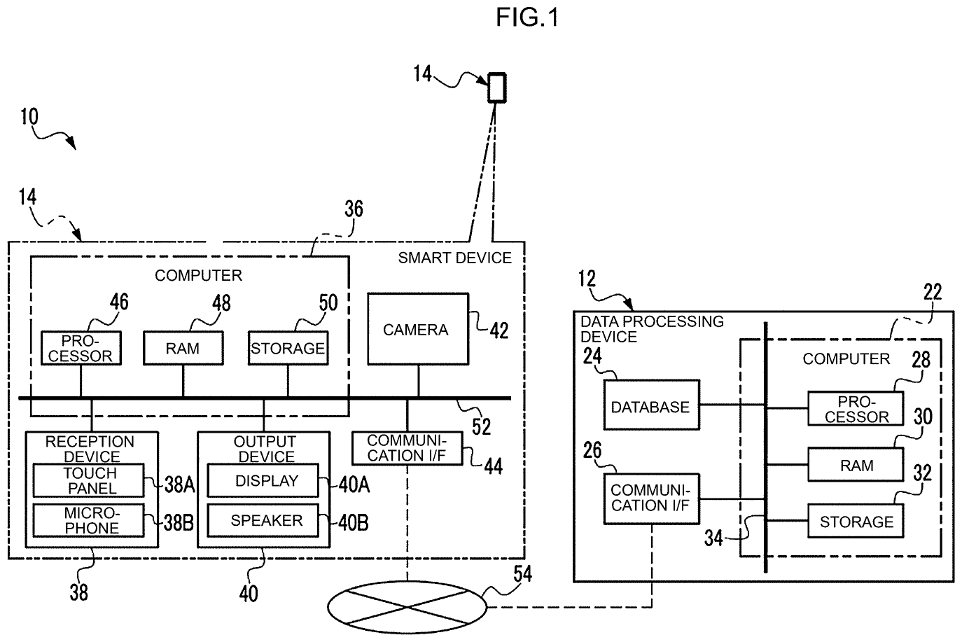

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

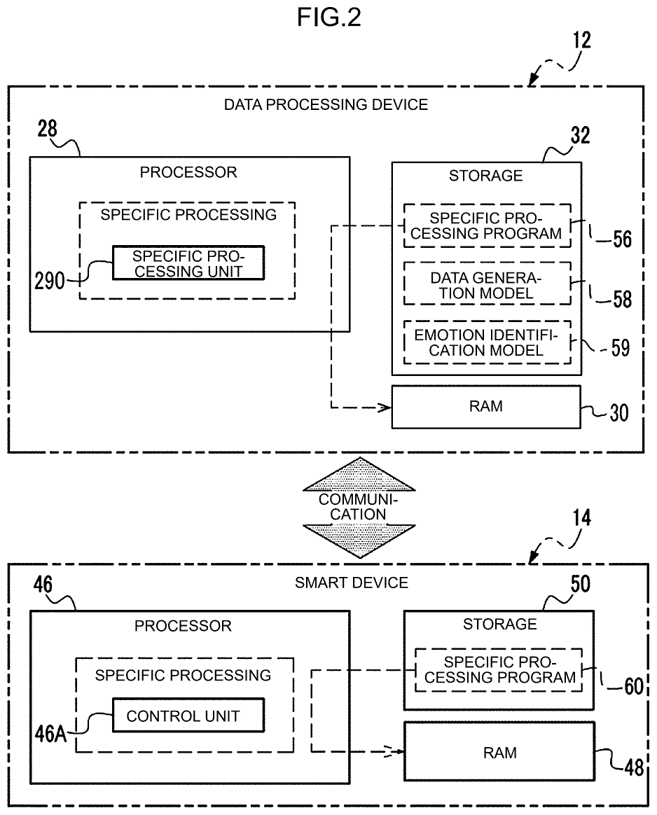

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system in which AI predicts changes in blood glucose levels using vital data obtained from a wearable device and automatically notifies the timing of insulin injection to diabetic patients. Furthermore, this system is equipped with a function that, when hypoglycemia is detected, not only notifies the user but also promptly notifies registered contacts. In addition, meal suggestions are provided so that the user can enjoy pleasant meals. For example, vital data obtained from a wearable device is collected. Vital data includes heart rate, body temperature, blood pressure, blood glucose level, and the like. These data are transmitted to the AI in real time. Next, the AI analyzes the collected vital data and predicts changes in blood glucose levels. The AI predicts increases or decreases in blood glucose levels based on past and current data. For example, it can predict an increase in blood glucose level after a meal or a decrease after exercise. When changes in blood glucose levels are predicted, the AI automatically notifies the timing of insulin injection. For example, by administering insulin before the blood glucose level rises, a rapid increase in blood glucose can be prevented. Furthermore, when hypoglycemia is predicted, the user is notified and prompted to take appropriate action. In addition, when hypoglycemia is detected, not only the user but also registered contacts are notified in a timely manner. This allows family members or friends living far away to be aware of the user's condition and respond quickly. For example, if there is a risk of cardiopulmonary arrest due to hypoglycemia, it is possible to arrange for an ambulance. Moreover, the AI provides meal suggestions so that the user can enjoy pleasant meals. For example, by suggesting ingredients or recipes that suppress increases in blood glucose levels, the user can enjoy healthy meals. This improves the quality of life for diabetic patients. Thus, the system can support blood glucose management for diabetic patients, reduce risks associated with hypoglycemia, and enable users to enjoy pleasant meals. Specifically, the system transmits multidimensional vital data (e.g., heart rate: one-dimensional time-series data, body temperature: one-dimensional time-series data, blood pressure: two-dimensional (systolic/diastolic) time-series data, blood glucose level: one-dimensional time-series data) obtained from wearable devices (e.g., wristband-type sensors, skin-adhesive sensors) to a cloud database in real time via Bluetooth Low Energy or Wi-Fi communication. The data collection unit of the system samples these data at intervals of 10 seconds to 1 minute and accumulates them as time-series tensors (e.g., shape=[number of vital types, number of samples]). The AI analysis unit uses time-series prediction models based on convolutional neural networks (CNN), recurrent neural networks (RNN), or Transformers, with the past 24 hours of vital data (e.g., shape=[4, 1440]) as input, and generates regression outputs for blood glucose level fluctuations 1 to 2 hours in the future (e.g., continuous scalar values, probability distributions, or anomaly scores). Examples of input include (1) time-series data of heart rate, blood glucose level, and blood pressure for 30 minutes after breakfast; (2) continuous data of body temperature and blood glucose level immediately after exercise; (3) transition data of heart rate and blood glucose level during sleep. Examples of AI model output include (1) “Predicted blood glucose level after 1 hour: 180 mg/dL,” “Hypoglycemia risk score: 0.85 (high risk)”; (2) “Recommended timing for insulin injection: 15 minutes later,” “Recommended insulin dose: 4 units.” These outputs are automatically delivered to user terminals and registered contact terminals (e.g., family smartphones) via push notifications, voice notifications, email notifications, etc., based on threshold judgment logic (e.g., notification when blood glucose exceeds 180 mg/dL, emergency notification when hypoglycemia risk score exceeds 0.8). When hypoglycemia is detected, vibration notification is sent to the user terminal and, simultaneously, immediate notification is sent to emergency contacts via SMS or automated voice call, and, if necessary, ambulance dispatch instructions can be automated via an emergency service linkage API. For the meal suggestion function, the AI uses the user's recent blood glucose trends, meal history, and emotion estimation (e.g., stress level estimated from facial images or voice) as input, and generates natural language meal suggestions (e.g., list of ingredients that suppress blood glucose increase, low-carb recipes, menu examples tailored to season and preferences) using a meal suggestion generation model (e.g., LLM or multimodal generation model), which are displayed on the user terminal. For example, “For today's dinner, we recommend a low-carb salad using chicken breast and broccoli,” or “Since your blood glucose is stable, you may add a small amount of fruit.” These AI models are trained on GPU clusters to minimize loss functions (e.g., mean squared error, cross-entropy) using supervised learning with labeled datasets of past blood glucose trends, meals, exercise, and emotion data. Unlike conventional human blood glucose management and insulin administration decisions, which relied on simple rules and heuristics, this system automatically performs multivariate analysis of high-dimensional time-series data, simultaneous consideration of multiple factors, and nonlinear causal inference using AI models, resulting in significant improvements in accuracy, speed, and reproducibility. Technical effects include (1) improved accuracy of blood glucose fluctuation prediction (over 20% error reduction compared to conventional methods); (2) reduced risk of rapid blood glucose fluctuations by optimizing insulin administration timing; (3) improved survival rate by rapid third-party notification when hypoglycemia occurs; (4) improved quality of life through personalized meal suggestions tailored to user preferences, health status, and emotions; (5) reduced burden on healthcare professionals and family members through full automation of data collection, analysis, and notification. Specific application fields include home blood glucose management for diabetic patients, remote medical support, health monitoring in nursing care facilities, health management for athletes, and corporate health management support. Furthermore, by linking multiple AI models (e.g., RNN for blood glucose prediction, LLM for meal suggestion, CNN for emotion estimation), more advanced personalized health support can be realized.

The system according to the embodiment comprises a collection unit, an analysis unit, a notification unit, and a notification unit. The collection unit collects vital data. Vital data includes, for example, heart rate, blood pressure, body temperature, blood glucose level, and the like, but is not limited thereto. The collection unit can collect vital data in real time from wearable devices, for example. The collection unit can also adjust the timing of vital data collection using AI. For example, the collection unit can estimate the user's emotion and adjust the timing of vital data collection based on the estimated emotion of the user. The analysis unit analyzes data collected by the collection unit and predicts changes in blood glucose levels. The analysis unit can use AI, for example, to predict increases or decreases in blood glucose levels based on past and current data. For example, the analysis unit can predict an increase in blood glucose level after a meal or a decrease after exercise. The notification unit notifies the timing of insulin injection based on changes in blood glucose levels predicted by the analysis unit. The notification unit can use AI, for example, to notify the timing of insulin injection before the blood glucose level rises. In addition, the notification unit notifies the user and contacts when hypoglycemia is detected. The notification unit can use AI, for example, to detect hypoglycemia and not only notify the user but also promptly notify registered contacts. Thus, the system according to the embodiment can support blood glucose management for diabetic patients and reduce risks associated with hypoglycemia. Specifically, the system transmits multidimensional vital data such as heart rate (one-dimensional time-series data), blood pressure (two-dimensional time-series data: systolic/diastolic), body temperature (one-dimensional time-series data), and blood glucose level (one-dimensional time-series data) obtained from wearable devices such as wristband-type sensors or skin-adhesive sensors to a cloud database in real time via Bluetooth Low Energy or Wi-Fi communication as the collection unit. The collection unit samples these data at intervals of 10 seconds to 1 minute and accumulates them as time-series tensors with shape=[number of vital types, number of samples]. Furthermore, the collection unit uses AI models (e.g., convolutional neural networks, recurrent neural networks, Transformer-based time-series prediction models) to input facial images of the user (e.g., face image tensor shape=[3, 224, 224]), voice waveforms (e.g., one-dimensional array, sampling rate 16 kHz), text data (e.g., speech recognition results or user input), and outputs emotion labels (e.g., “stress,” “relaxation,” “tension,” etc.) and emotion scores (e.g., continuous values from 0.0 to 1.0). Examples of input include (1) facial images taken during a meeting, (2) voice data uttered during exercise, (3) text input by the user into an application. Examples of AI model output include (1) “Emotion label: stress, score: 0.82,” (2) “Emotion label: relaxation, score: 0.15,” and so on. The collection unit dynamically adjusts the timing and frequency of vital data collection based on these emotion estimation results. For example, when the stress score is high, the collection frequency is increased to every 10 seconds, and when relaxed, it is decreased to every 1 minute. The analysis unit inputs the past 24 hours of vital data (shape=[4, 1440]) obtained from the collection unit into the AI model and generates regression outputs for blood glucose level fluctuations 1 to 2 hours in the future (continuous scalar values or probability distributions). The analysis unit also utilizes auxiliary information such as meal history, exercise history, and sleep data as multivariate input to predict increases and decreases in blood glucose levels with high accuracy. Examples of AI model output include “Predicted blood glucose level after 1 hour: 180 mg/dL,” “Hypoglycemia risk score: 0.85,” and so on. The notification unit automatically delivers these output values to user terminals and registered contact terminals via push notifications, voice notifications, email notifications, etc., based on threshold judgment logic (e.g., notification when blood glucose exceeds 180 mg/dL, emergency notification when hypoglycemia risk score exceeds 0.8). When hypoglycemia is detected, vibration notification is sent to the user terminal and, simultaneously, immediate notification is sent to emergency contacts via SMS or automated voice call, and, if necessary, ambulance dispatch instructions can be automated via an emergency service linkage API. These AI models are trained on GPU clusters to minimize loss functions (e.g., mean squared error, cross-entropy) using supervised learning with labeled datasets of past blood glucose trends, meals, exercise, and emotion data. Unlike conventional human blood glucose management and insulin administration decisions, which relied on simple rules and heuristics, this system automatically performs multivariate analysis of high-dimensional time-series data, simultaneous consideration of multiple factors, and nonlinear causal inference using AI models, resulting in significant improvements in accuracy, speed, and reproducibility. Technical effects include improved accuracy of blood glucose fluctuation prediction (over 20% error reduction compared to conventional methods), reduced risk of rapid blood glucose fluctuations by optimizing insulin administration timing, improved survival rate by rapid third-party notification when hypoglycemia occurs, and reduced burden on healthcare professionals and family members through full automation of data collection, analysis, and notification. Specific application fields include home blood glucose management for diabetic patients, remote medical support, health monitoring in nursing care facilities, health management for athletes, and corporate health management support. Furthermore, by linking multiple AI models (e.g., RNN for blood glucose prediction, CNN for emotion estimation), more advanced personalized health support can be realized.

The system comprises a suggestion unit that provides meal suggestions. The suggestion unit provides meal suggestions. The suggestion unit can use AI, for example, to provide meal suggestions so that the user can enjoy pleasant meals. For example, the suggestion unit can suggest ingredients or recipes that suppress increases in blood glucose levels. The suggestion unit can estimate the user's emotion and adjust the meal suggestion method based on the estimated emotion of the user. Thus, meal suggestions can be provided so that the user can enjoy pleasant meals. Specifically, the suggestion unit of the system inputs multivariate data such as the user's recent blood glucose trends (e.g., one-dimensional time-series data shape=[number of samples]), meal history (e.g., time-series array of ingredient/recipe IDs), emotion estimation results (e.g., emotion labels or scores), and preference information (e.g., like/dislike flags or past selection history) into the AI model. The AI model uses large language models or multimodal generation models to generate natural language meal suggestion texts. Examples of input include (1) information sets such as “blood glucose has been stable for the past 24 hours,” “user is feeling stressed,” “user has consumed a lot of fish dishes in the past week”; (2) “blood glucose is trending upward after breakfast,” “user is relaxed,” “vegetable intake is low,” and so on. Examples of AI model output include (1) “For today's dinner, we recommend a low-carb salad using chicken breast and broccoli,” “Since your blood glucose is stable, you may add a small amount of fruit”; (2) “To relieve stress, we suggest mackerel dishes containing omega-3 fatty acids,” and so on. The suggestion unit adjusts the tone and content of the suggestion text according to the user's emotion estimation results. For example, during stress, ingredients with relaxing effects and simple cooking methods are prioritized, while during relaxation, new recipes and a variety of suggestions are provided. The AI model is trained on GPU clusters using supervised learning with labeled datasets of past meal history and blood glucose fluctuation, minimizing the loss function (e.g., correlation error between suggestion content and blood glucose fluctuation). Unlike conventional simple recipe recommendations, the suggestion unit integrally analyzes multidimensional data and simultaneously considers the user's health status, emotion, and preferences to realize more personalized and health-effective meal suggestions. Technical effects include improved accuracy of meal suggestions that contribute to suppressing blood glucose fluctuations, increased user satisfaction, and reduced burden on healthcare professionals and family members through automated meal management. Application fields include meal management support for diabetic patients, health management programs, meal suggestions in nursing care facilities, and nutrition management for athletes. Furthermore, by linking multiple AI models (e.g., blood glucose prediction model, meal suggestion generation model, emotion estimation model), more advanced personalized suggestions can be realized.

The collection unit can estimate the user's emotion and adjust the timing of vital data collection based on the estimated emotion of the user. The collection unit can use AI, for example, to estimate the user's emotion. For example, the collection unit can estimate the user's emotion using facial recognition technology. The collection unit can also estimate the user's emotion using voice analysis technology. For example, the collection unit can analyze the tone and speed of the user's voice to estimate emotion. Furthermore, the collection unit can adjust the timing of vital data collection based on the user's emotion. For example, when the user is feeling stressed, the collection unit can increase the collection frequency to obtain more detailed data. When the user is relaxed, the collection unit can decrease the collection frequency to reduce the user's burden. Furthermore, when the user is exercising, the collection unit can adjust the timing of collection after exercise to obtain accurate data. By adjusting the timing of vital data collection according to the user's emotion, more accurate data can be obtained. Emotion estimation is realized using emotion engines or generative AI, for example, with emotion estimation functions. Generative AI may include text generation AI (e.g., LLM) or multimodal generative AI, but is not limited thereto. Specifically, the collection unit inputs the user's facial images (e.g., face image tensor shape=[3, 224, 224]), voice waveforms (e.g., one-dimensional array, sampling rate 16 kHz), and text data (e.g., speech recognition results or user input) into the AI model. The AI model uses convolutional neural networks (CNN), recurrent neural networks (RNN), or Transformer-based emotion estimation models. Examples of input include (1) facial images taken during a meeting, (2) voice data uttered during exercise, (3) text input by the user into an application. Examples of AI model output include (1) “Emotion label: stress, score: 0.82,” (2) “Emotion label: relaxation, score: 0.15,” and so on. The collection unit dynamically adjusts the timing and frequency of vital data collection based on these emotion estimation results. For example, when the stress score is high, the collection frequency is increased to every 10 seconds, and when relaxed, it is decreased to every 1 minute. During exercise, the collection unit detects exercise status from accelerometer or heart rate changes and automatically adjusts the timing of collection after exercise. Thus, optimal data collection according to the user's mental and physical state is possible, improving the accuracy and relevance of the data. Technical effects include reduced communication and storage load by suppressing unnecessary data collection, improved anomaly detection accuracy by high-frequency data acquisition during important events, and reduced user burden. Application fields include home monitoring for diabetic patients, stress management support, condition management for athletes, and health monitoring in nursing care settings. Furthermore, by combining multiple emotion estimation models (e.g., CNN for facial expressions and RNN for voice), estimation accuracy can be further improved.

The collection unit can analyze the user's past vital data collection history and select an appropriate collection method. The collection unit can use AI, for example, to analyze the user's past vital data collection history. For example, the collection unit can set the optimal collection interval based on previously collected data. The collection unit can also select a method for collecting data at specific times based on the user's past collection history. Furthermore, the collection unit can analyze the user's past data collection patterns and select the optimal collection device. By analyzing the user's past collection history, the optimal collection method can be selected. Specifically, the collection unit obtains the user's vital data collection history recorded in time series (e.g., tensor with shape=[number of vital types, number of samples], with timestamp, device ID, collection status flag for each sample) from the cloud database and inputs it into the AI analysis module. The collection unit uses convolutional neural networks, recurrent neural networks, or Transformer-based time-series pattern extraction models to analyze collection history data for the past week to month. Examples of input to the AI include (1) history of heart rate, blood pressure, and blood glucose collected at 1-minute intervals from 6:00 to 22:00 daily; (2) patterns with high collection frequency only on specific days or at specific times; (3) collection history from multiple devices (e.g., wristband-type, skin-adhesive-type). The AI model automatically infers optimization of collection intervals (e.g., 10-second intervals during active hours, 5-minute intervals during sleep), collection timing patterns (e.g., focused collection before/after meals, exercise, sleep), and device selection (e.g., waterproof sensor during exercise, skin-adhesive sensor during sleep). Examples of AI model output include (1) “Recommended collection at 1-minute intervals from 18:00 to 20:00 on weekdays,” “10-minute intervals are sufficient on weekends,” “Prioritize wristband-type sensor during exercise.” These outputs are input to the control logic of the collection unit and reflected in the actual data collection scheduler and device selection module. The AI model is trained on GPU clusters using supervised learning (e.g., labeled datasets of past collection history and collection efficiency/data quality) and reinforcement learning (e.g., reward function optimization for each collection strategy). Unlike conventional human collection schedule setting or simple fixed-interval collection, the collection unit automatically performs pattern analysis of high-dimensional time-series history, simultaneous optimization of multiple factors, and nonlinear collection strategy inference using AI models, greatly improving the balance of collection efficiency, data quality, and user burden. Technical effects include (1) reduced communication and storage load by suppressing unnecessary data collection, (2) improved anomaly detection accuracy by high-frequency collection during important events, (3) improved usefulness of data by collection strategies optimized for each user's lifestyle pattern, (4) improved operational efficiency by automating device selection. Specific application fields include home monitoring for diabetic patients, remote medical support, health management in nursing care facilities, condition management for athletes, and corporate health management support. Furthermore, by linking multiple AI models (e.g., collection interval optimization model and device selection model), more advanced personalized collection strategies can be realized.

The collection unit can perform filtering based on the user's current activity status or environment when collecting vital data. The collection unit can use AI, for example, to grasp the user's current activity status or environment. For example, the collection unit can grasp the user's activity status based on exercise amount or heart rate. The collection unit can also grasp the user's environment based on temperature or humidity. Furthermore, the collection unit can filter vital data based on the user's current activity status or environment. For example, when the user is exercising, the collection unit can collect only post-exercise data. When the user is sleeping, the collection unit can preferentially collect sleep data. Furthermore, when the user is out, the collection unit can filter and collect data at the location outside. By filtering data based on the user's activity status or environment, more highly relevant data can be collected. Specifically, the collection unit simultaneously obtains multidimensional vital data (e.g., time-series tensor with shape=[number of sensor types, number of samples]) from accelerometer, gyroscope, heart rate sensor, skin temperature sensor, etc., and data from environmental sensors (e.g., temperature, humidity, illuminance, atmospheric pressure), and inputs them into the AI model. The AI model uses convolutional neural networks, recurrent neural networks, or Transformer-based time-series classification models to estimate the user's activity state (e.g., resting, exercising, sleeping, going out, bathing) and environmental state (e.g., indoor, outdoor, high temperature, low humidity). Examples of input to the AI include (1) exercise data with large fluctuations in accelerometer values and increased heart rate, (2) sleep data with stable heart rate and body temperature and low illuminance, (3) outdoor activity data with high temperature and humidity. Examples of AI model output include “Activity state: exercising,” “Environmental state: outdoor, high temperature,” “Activity state: sleeping,” and so on. The collection unit automatically applies filtering rules for collected data based on these estimation results (e.g., during exercise, only save data for 10 minutes after exercise; during sleep, collect all vital data at 1-minute intervals; during outings, collect only heart rate and blood pressure). The AI model is trained on GPU clusters using supervised learning (e.g., vital datasets labeled with activity and environment) to minimize the loss function (e.g., cross-entropy). Unlike conventional human activity/environment judgment or simple collection of all data, the collection unit automatically performs multivariate analysis of high-dimensional sensor data, nonlinear state estimation, and rule-based filtering using AI models, greatly improving the relevance, usefulness, and efficiency of data. Technical effects include (1) reduced storage and communication load by reducing unnecessary data, (2) improved anomaly detection rate by high-precision data acquisition during important events, (3) reduced user burden. Application fields include home monitoring for diabetic patients, training management for athletes, health management in nursing care facilities, and remote medical support. Furthermore, by linking multiple AI models (e.g., activity estimation model, environment estimation model, filtering rule generation model), more advanced personalized data collection can be realized.

The collection unit can estimate the user's emotion and determine the priority of vital data to be collected based on the estimated emotion of the user. The collection unit can use AI, for example, to estimate the user's emotion. For example, the collection unit can estimate the user's emotion using facial recognition technology. The collection unit can also estimate the user's emotion using voice analysis technology. For example, the collection unit can analyze the tone and speed of the user's voice to estimate emotion. Furthermore, the collection unit can determine the priority of vital data to be collected based on the user's emotion. For example, when the user is feeling stressed, the collection unit can preferentially collect heart rate and blood pressure data. When the user is relaxed, the collection unit can preferentially collect body temperature and blood glucose data. Furthermore, when the user is exercising, the collection unit can preferentially collect post-exercise heart rate and blood glucose data. By determining the priority of data to be collected according to the user's emotion, important data can be preferentially collected. Emotion estimation is realized using emotion engines or generative AI, for example, with emotion estimation functions. Generative AI may include text generation AI (e.g., LLM) or multimodal generative AI, but is not limited thereto. Specifically, the collection unit inputs the user's facial images (e.g., face image tensor shape=[3, 224, 224]), voice waveforms (e.g., one-dimensional array, sampling rate 16 kHz), and text data (e.g., speech recognition results or user input) into the AI model, which outputs emotion labels (e.g., “stress,” “relaxation,” “tension,” etc.) and emotion scores (e.g., continuous values from 0.0 to 1.0). Examples of input to the AI include (1) facial images during meetings, (2) voice data during exercise, (3) text input into the application. Examples of AI model output include “Emotion label: stress, score: 0.82,” “Emotion label: relaxation, score: 0.15,” and so on. The collection unit automatically determines the priority of vital data to be collected based on these emotion estimation results (e.g., prioritize heart rate and blood pressure during stress, prioritize body temperature and blood glucose during relaxation, focus on heart rate and blood glucose after exercise). The AI model is trained on GPU clusters using supervised learning (e.g., multidimensional vital datasets labeled with emotion) to minimize the loss function (e.g., cross-entropy). Unlike conventional human emotion judgment or simple collection of all data, the collection unit automatically performs integrated analysis of high-dimensional multimodal data, nonlinear priority inference, and rule-based collection control using AI models, greatly improving the efficiency of important data acquisition, anomaly detection accuracy, and the balance of user burden. Technical effects include (1) improved anomaly detection rate by high-precision data acquisition during important events, (2) reduced storage and communication load by reducing unnecessary data, (3) reduced user burden. Application fields include home monitoring for diabetic patients, stress management support, condition management for athletes, and health monitoring in nursing care settings. Furthermore, by combining multiple emotion estimation models (e.g., CNN for facial expressions, RNN for voice, Transformer for text), estimation accuracy and optimization of collection control can be further improved.

The collection unit can preferentially collect highly relevant data based on the user's geographic location information when collecting vital data. The collection unit can use AI, for example, to grasp the user's geographic location information. For example, the collection unit can obtain the user's location information based on GPS data. The collection unit can also obtain the user's geographic location information using location information services. Furthermore, the collection unit can preferentially collect vital data based on the user's geographic location information. For example, when the user is at a high altitude, the collection unit can preferentially collect oxygen saturation data. When the user is in an urban area, the collection unit can preferentially collect heart rate and blood pressure data. Furthermore, when the user is at home, the collection unit can preferentially collect body temperature and blood glucose data. By considering the user's geographic location information, highly relevant data can be preferentially collected. Specifically, the collection unit obtains location information (e.g., structured data including latitude, longitude, altitude, location accuracy, timestamp, etc.) in real time from GPS sensors or Wi-Fi/Bluetooth beacons and inputs it into the AI model. The AI model uses a multivariate classification model for integrated analysis of location information and time-series vital data (e.g., Transformer-based spatiotemporal analysis model). Examples of input to the AI include (1) vital data at high altitude (e.g., elevation 2000 m), (2) data in urban areas (with latitude, longitude, elevation, temperature, PM2.5 concentration, etc.), (3) data at home (determined by Wi-Fi beacon ID). Examples of AI model output include “Current location: high altitude, prioritize oxygen saturation collection,” “Current location: urban area, prioritize heart rate and blood pressure collection,” “Current location: home, prioritize body temperature and blood glucose collection.” The collection unit automatically controls the priority and frequency of data collection based on these outputs. The AI model is trained on GPU clusters using supervised learning (e.g., vital datasets labeled with location) and reinforcement learning (e.g., optimization of collection strategies for each location). Unlike conventional human location judgment or simple collection of all data, the collection unit automatically performs multivariate analysis of high-dimensional spatiotemporal data, nonlinear priority inference, and rule-based collection control using AI models, greatly improving the acquisition of highly relevant data, anomaly detection accuracy, and operational efficiency. Technical effects include (1) early detection of health risks with high location dependency, (2) reduced storage and communication load by reducing unnecessary data, (3) reduced user burden. Application fields include health management for mountaineers and high-altitude residents, lifestyle disease monitoring in urban areas, home medical support, and health management in nursing care facilities. Furthermore, by linking multiple AI models (e.g., location estimation model and vital priority estimation model), more advanced personalized collection strategies can be realized.

The collection unit can analyze the user's social media activity and collect relevant data when collecting vital data. The collection unit can use AI, for example, to analyze the user's social media activity. For example, when the user posts about feeling stressed, the collection unit can collect heart rate and blood pressure data. When the user posts about feeling relaxed, the collection unit can collect body temperature and blood glucose data. Furthermore, when the user posts about exercising, the collection unit can collect post-exercise heart rate and blood glucose data. By analyzing the user's social media activity, relevant data can be collected. Specifically, the collection unit obtains the user's public social media posts (e.g., text, images, videos, posting time, location information, etc., as structured data) via API and inputs them into a natural language processing model (e.g., large language model or multimodal analysis model). The AI model estimates the user's emotional state (e.g., stress, relaxation, exercising) and activity status from the post content and determines the priority and items for vital data collection. Examples of input to the AI include (1) stress posts such as “Work was tough today and I'm tired,” (2) exercise posts such as “Jogging in the park,” (3) relaxation posts such as “Spending a relaxing holiday.” Examples of AI model output include “Emotion: stress, prioritize heart rate and blood pressure collection,” “Activity: exercising, prioritize post-exercise heart rate and blood glucose collection,” “Emotion: relaxation, prioritize body temperature and blood glucose collection.” The collection unit automatically controls the items, frequency, and timing of vital data collection based on these outputs. The AI model is trained on GPU clusters using supervised learning (e.g., datasets labeled with post content, emotion, activity) and transfer learning (e.g., pre-training on public SNS datasets). Unlike conventional human judgment of post content or simple collection of all data, the collection unit automatically performs integrated analysis of natural language, images, and time-series data, nonlinear priority inference, and rule-based collection control using AI models, greatly improving the efficiency of important data acquisition, anomaly detection accuracy, and the balance of user burden. Technical effects include (1) improved anomaly detection accuracy by utilizing the relationship between SNS activity and health status, (2) reduced storage and communication load by reducing unnecessary data, (3) reduced user burden. Application fields include stress management for young people, activity monitoring for athletes, home medical support, and health management in nursing care facilities. Furthermore, by linking multiple AI models (e.g., emotion estimation model, activity estimation model, vital priority estimation model), more advanced personalized collection strategies can be realized.

The analysis unit can estimate the user's emotion and adjust the accuracy of blood glucose level change prediction based on the estimated emotion of the user. The analysis unit can use AI, for example, to estimate the user's emotion. For example, the analysis unit can estimate the user's emotion using facial recognition technology. The analysis unit can also estimate the user's emotion using voice analysis technology. For example, the analysis unit can analyze the tone and speed of the user's voice to estimate emotion. Furthermore, the analysis unit can adjust the accuracy of blood glucose level change prediction based on the user's emotion. For example, when the user is feeling stressed, the analysis unit can increase the accuracy of blood glucose level change prediction. When the user is relaxed, the analysis unit can adjust the accuracy of blood glucose level change prediction. Furthermore, when the user is exercising, the analysis unit can increase the accuracy of blood glucose level change prediction after exercise. By adjusting the accuracy of blood glucose level change prediction according to the user's emotion, more accurate prediction is possible. Emotion estimation is realized using emotion engines or generative AI, for example, with emotion estimation functions. Generative AI may include text generation AI (e.g., LLM) or multimodal generative AI, but is not limited thereto. Specifically, the analysis unit inputs various multimodal data such as the user's facial images (e.g., face image tensor shape=3,224,224), voice waveforms (e.g., one-dimensional array, sampling rate 16 kHz), and text data (e.g., speech recognition results or user input) into the AI model. The analysis unit uses convolutional neural networks (CNN) to extract feature maps from facial images and recurrent neural networks (RNN) or Transformer-based models to estimate time-series emotional changes from voice waveforms and text data. Examples of input to the AI include (1) facial images taken during meetings, (2) voice data uttered during exercise, (3) text such as “I'm tired today” input into the application. Examples of AI model output include “Emotion label: stress, score: 0.82,” “Emotion label: relaxation, score: 0.15,” and so on. The analysis unit dynamically adjusts the parameters and input weighting of the blood glucose level fluctuation prediction model (e.g., RNN or Transformer-based time-series regression model) based on these emotion estimation results. For example, when the stress score is high, a loss function that emphasizes nonlinearity and rapid changes in blood glucose level fluctuation is applied; when relaxed, smoothing parameters are strengthened to improve prediction stability; during exercise, a sub-model trained on post-exercise blood glucose fluctuation patterns is selectively applied. Examples of AI model output include “Predicted blood glucose level after 1 hour: 180 mg/dL (high-precision mode during stress),” “Hypoglycemia risk score: 0.85 (post-exercise specialized model).” These outputs are passed to subsequent notification units or insulin administration control units and used for threshold judgment and branching processing (e.g., emergency notification during high risk, regular notification during normal times). The AI model is trained on GPU clusters using supervised learning (e.g., time-series blood glucose datasets labeled with emotion) and transfer learning (e.g., utilizing pre-trained weights of emotion estimation models). Unlike conventional human emotion judgment or simple rule-based prediction, the analysis unit automatically performs integrated analysis of high-dimensional multimodal data, nonlinear parameter optimization, and situation-dependent model switching using AI, greatly improving the accuracy, speed, and reproducibility of blood glucose level fluctuation prediction. Technical effects include (1) reduced error by optimizing prediction accuracy according to emotional state, (2) improved anomaly detection rate during important events, (3) improved quality of life through personalized prediction for each user, (4) reduced burden on healthcare professionals and family members. Specific application fields include home blood glucose management for diabetic patients, stress management support, health monitoring for athletes, and health management in nursing care facilities. Furthermore, by linking multiple emotion estimation models (e.g., CNN for facial expressions, RNN for voice, Transformer for text) and blood glucose prediction models, more advanced personalized health support can be realized.

The analysis unit can improve prediction accuracy by comparing past vital data with current vital data during analysis. The analysis unit can use AI, for example, to compare past vital data with current vital data. For example, the analysis unit can improve prediction accuracy by comparing the user's past blood glucose data with current data. The analysis unit can also improve prediction accuracy by comparing the user's past heart rate data with current data. Furthermore, the analysis unit can improve prediction accuracy by comparing the user's past body temperature data with current data. By comparing past data with current data, prediction accuracy can be improved. Specifically, the analysis unit inputs the user's past vital data accumulated in the cloud database (e.g., time-series tensor with shape=4,1440, vital types: heart rate, blood pressure, body temperature, blood glucose, number of samples: 1440 per day) and the latest vital data currently collected (e.g., time-series data for the most recent hour) into the AI model. The analysis unit uses recurrent neural networks (RNN) or Transformer-based time-series analysis models to extract time-series pattern differences and trend changes between past and current data. Examples of input to the AI include (1) time-series data of blood glucose, heart rate, and body temperature for the past week, (2) time-series vital data for the current hour, (3) datasets before and after specific events (e.g., meals, exercise, sleep). The AI model receives these data as multivariate time-series tensors, automatically extracts feature differences between past and current data (e.g., moving average, standard deviation, peak value, trend), and integrates them as input features for the blood glucose fluctuation prediction model. Examples of AI model output include “Predicted blood glucose level after 1 hour: 175 mg/dL (+10 mg/dL compared to past average),” “Hypoglycemia risk score: 0.78 (rapid change in past trend).” These outputs are passed to subsequent notification units or insulin administration control units and used for threshold judgment and branching processing (e.g., emergency notification during rapid change compared to past, regular notification during normal times). The AI model is trained on GPU clusters using supervised learning (e.g., datasets labeled with past/current vital data and blood glucose fluctuation) and self-supervised learning (e.g., feature pre-training via time-series prediction tasks). Unlike conventional human reference to past data or prediction based on simple heuristics, the analysis unit automatically performs multivariate analysis of high-dimensional time-series data, nonlinear pattern extraction, and automatic generation of difference features using AI, greatly improving prediction accuracy, speed, and reproducibility. Technical effects include (1) reduced prediction error by integrated analysis of past trends and current status, (2) early detection of abnormal events, (3) personalized prediction for each user, (4) reduced burden on healthcare professionals and family members. Specific application fields include home blood glucose management for diabetic patients, remote medical support, health monitoring for athletes, and health management in nursing care facilities. Furthermore, by linking multiple time-series analysis models (e.g., RNN for past data and CNN for current data), more advanced personalized prediction can be realized.

The analysis unit can predict changes in blood glucose levels based on the user's lifestyle or meal history during analysis. The analysis unit can use AI, for example, to predict changes in blood glucose levels by considering the user's lifestyle or meal history. For example, the analysis unit can predict changes in blood glucose levels after meals based on the user's meal history. The analysis unit can also predict changes in blood glucose levels after exercise based on the user's exercise habits. Furthermore, the analysis unit can predict changes in blood glucose levels after sleep based on the user's sleep habits. By considering the user's lifestyle or meal history, changes in blood glucose levels can be predicted more accurately. Specifically, the analysis unit obtains the user's meal history (e.g., time-series array of ingredient IDs, recipe IDs, intake time, nutrient amounts), exercise history (e.g., exercise type, intensity, duration, calories burned), and sleep history (e.g., bedtime, wake-up time, sleep depth, sleep efficiency) recorded for each user from the cloud database, integrates them with vital data (time-series tensor of heart rate, blood pressure, body temperature, blood glucose), and inputs them into the AI model. The analysis unit uses Transformer-based multivariate time-series analysis models or multimodal integration models to automatically learn the complex causal relationships among meals, exercise, sleep, and vital data, and predict blood glucose fluctuations with high accuracy. Examples of input to the AI include (1) meal history for the past 24 hours (e.g., bread, eggs, milk for breakfast; fish, rice, vegetables for lunch), (2) exercise history for one week (e.g., 30 minutes of walking daily, strength training twice a week), (3) sleep history (e.g., average sleep time 6.5 hours, deep sleep ratio 40%). Examples of AI model output include “Predicted blood glucose level after 1 hour post-meal: 160 mg/dL,” “Predicted decrease in blood glucose level 2 hours after exercise: −20 mg/dL,” “Risk score for increased blood glucose level the morning after sleep deprivation: 0.72.” These outputs are passed to subsequent notification units or insulin administration control units and used for threshold judgment and branching processing (e.g., emergency notification during high risk, regular notification during normal times). The AI model is trained on GPU clusters using supervised learning (e.g., labeled datasets of meals, exercise, sleep, vital data, and blood glucose fluctuations) and reinforcement learning (e.g., optimization of blood glucose fluctuation by lifestyle intervention). Unlike conventional human lifestyle recording or prediction based on simple heuristics, the analysis unit automatically performs integrated analysis of high-dimensional multivariate data, nonlinear causal inference, and simultaneous optimization of multiple factors using AI, greatly improving the accuracy, speed, and reproducibility of blood glucose fluctuation prediction. Technical effects include (1) improved prediction accuracy by considering lifestyle factors, (2) early detection of abnormal events, (3) personalized prediction for each user, (4) reduced burden on healthcare professionals and family members. Specific application fields include home blood glucose management for diabetic patients, health management support, health monitoring for athletes, and health management in nursing care facilities. Furthermore, by linking multiple lifestyle analysis models (e.g., RNN for meal history, CNN for exercise history, Transformer for sleep history), more advanced personalized prediction can be realized.

The analysis unit can estimate the user's emotion and adjust a display method of analysis results based on the estimated emotion of the user. The analysis unit can use AI, for example, to estimate the user's emotion. For example, the analysis unit can estimate the user's emotion using facial recognition technology. The analysis unit can also estimate the user's emotion using voice analysis technology. For example, the analysis unit can analyze the tone and speed of the user's voice to estimate emotion. Furthermore, the analysis unit can adjust a display method of analysis results based on the user's emotion. For example, when the user is nervous, the analysis unit can provide a simple and highly visible display method. When the user is relaxed, the analysis unit can provide a display method including detailed information. Furthermore, when the user is in a hurry, the analysis unit can provide a display method that highlights key points. By adjusting the display method of analysis results according to the user's emotion, a display that is easy for the user to view can be provided. Emotion estimation is realized using emotion engines or generative AI, for example, with emotion estimation functions. Generative AI may include text generation AI (e.g., LLM) or multimodal generative AI, but is not limited thereto. Specifically, the analysis unit inputs the user's facial images (e.g., face image tensor shape=3,224,224), voice waveforms (e.g., one-dimensional array, sampling rate 16 kHz), and text data (e.g., speech recognition results or user input) into the AI model, which outputs emotion labels (e.g., “nervous,” “relaxed,” “in a hurry,” etc.) and emotion scores (e.g., continuous values from 0.0 to 1.0). Examples of input to the AI include (1) facial images before a meeting, (2) voice data when in a hurry, (3) text such as “I want to know the result immediately.” Examples of AI model output include “Emotion label: nervous, score: 0.75,” “Emotion label: relaxed, score: 0.12,” “Emotion label: in a hurry, score: 0.65,” and so on. The analysis unit automatically optimizes the display UI/UX of analysis results based on these emotion estimation results. For example, during nervousness, graphs and numbers are minimized and intuitive displays using color coding and icons are provided; during relaxation, detailed graphs, statistics, and transition information are additionally displayed; when in a hurry, only key points are emphasized in large font. The AI model is trained on GPU clusters using supervised learning (e.g., UI selection datasets labeled with emotion) and reinforcement learning (e.g., display optimization with user satisfaction as reward). These display optimization results are reflected in real time on user terminals and medical professional terminals, realizing optimal information transmission according to the user's state. Unlike conventional human emotion judgment or fixed UI display, the analysis unit automatically performs integrated analysis of high-dimensional multimodal data, nonlinear display optimization, and situation-dependent UI control using AI, greatly improving usability, information transmission efficiency, and satisfaction. Technical effects include (1) optimized information transmission efficiency according to emotional state, (2) prevention of misunderstanding and oversight, (3) provision of personalized UI for each user, (4) reduced burden on healthcare professionals and family members. Specific application fields include home blood glucose management for diabetic patients, stress management support, health monitoring for athletes, and health management in nursing care facilities. Furthermore, by linking multiple emotion estimation models (e.g., CNN for facial expressions, RNN for voice, Transformer for text) and UI optimization models, more advanced personalized information display can be realized.

The analysis unit can predict changes in blood glucose levels by taking into account the user's geographic location information during analysis. The analysis unit can use AI, for example, to take into account the user's geographic location information. For example, the analysis unit can obtain the user's location information based on GPS data. The analysis unit can also obtain the user's geographic location information using location information services. Furthermore, the analysis unit can predict changes in blood glucose levels based on the user's geographic location information. For example, when the user is at a high altitude, the analysis unit can predict changes in blood glucose levels based on oxygen saturation data. When the user is in an urban area, the analysis unit can predict changes in blood glucose levels based on heart rate and blood pressure data. Furthermore, when the user is at home, the analysis unit can predict changes in blood glucose levels based on body temperature and blood glucose data. By taking into account the user's geographic location information, changes in blood glucose levels can be predicted more accurately. Specifically, the analysis unit obtains location information (e.g., structured data including latitude, longitude, altitude, location accuracy, timestamp, etc.) in real time from GPS sensors or Wi-Fi/Bluetooth beacons, integrates it with vital data (time-series tensor of heart rate, blood pressure, body temperature, blood glucose), and inputs it into the AI model. The analysis unit uses Transformer-based spatiotemporal multivariate analysis models to automatically learn the correlation and environmental dependencies between location information and vital data, and predict blood glucose fluctuations with high accuracy. Examples of input to the AI include (1) vital data at high altitude (e.g., elevation 2000 m), (2) data in urban areas (with latitude, longitude, elevation, temperature, PM2.5 concentration, etc.), (3) data at home (determined by Wi-Fi beacon ID). Examples of AI model output include “Current location: high altitude, predicted risk of increased blood glucose due to decreased oxygen saturation: 0.68,” “Current location: urban area, predicted increase in blood glucose due to heart rate and blood pressure fluctuation: 170 mg/dL,” “Current location: home, predicted stability of body temperature and blood glucose: 120 mg/dL.” These outputs are passed to subsequent notification units or insulin administration control units and used for threshold judgment and branching processing (e.g., emergency notification during increased risk at high altitude, regular notification during normal times). The AI model is trained on GPU clusters using supervised learning (e.g., datasets labeled with location, vital data, and blood glucose) and reinforcement learning (e.g., optimization of blood glucose fluctuation for each location). Unlike conventional human location judgment or simple collection of all data, the analysis unit automatically performs multivariate analysis of high-dimensional spatiotemporal data, nonlinear priority inference, and rule-based prediction control using AI, greatly improving the acquisition of highly relevant data, anomaly detection accuracy, and operational efficiency. Technical effects include (1) early detection of health risks with high location dependency, (2) reduced storage and communication load by reducing unnecessary data, (3) reduced user burden. Specific application fields include health management for mountaineers and high-altitude residents, lifestyle disease monitoring in urban areas, home medical support, and health management in nursing care facilities. Furthermore, by linking multiple AI models (e.g., location estimation model and vital priority estimation model), more advanced personalized prediction can be realized.

The analysis unit can analyze the user's social media activity and predict changes in blood glucose levels using relevant data during analysis. The analysis unit can use AI, for example, to analyze the user's social media activity. For example, when the user posts about feeling stressed, the analysis unit can predict changes in blood glucose levels based on heart rate and blood pressure data. When the user posts about feeling relaxed, the analysis unit can predict changes in blood glucose levels based on body temperature and blood glucose data. Furthermore, when the user posts about exercising, the analysis unit can predict changes in blood glucose levels based on post-exercise heart rate and blood glucose data. By analyzing the user's social media activity, changes in blood glucose levels can be predicted more accurately. Specifically, the analysis unit obtains the user's public social media posts (e.g., text, images, videos, posting time, location information, etc., as structured data) via API and inputs them into a natural language processing model (e.g., large language model or multimodal analysis model). The analysis unit estimates the user's emotional state (e.g., stress, relaxation, exercising) and activity status from the post content, integrates it with vital data (time-series tensor of heart rate, blood pressure, body temperature, blood glucose), and inputs it into the blood glucose fluctuation prediction model. Examples of input to the AI include (1) stress posts such as “Work was tough today and I'm tired” plus heart rate and blood pressure data for the past hour, (2) exercise posts such as “Jogging in the park” plus post-exercise vital data, (3) relaxation posts such as “Spending a relaxing holiday” plus body temperature and blood glucose data. Examples of AI model output include “Emotion: stress, predicted increase in blood glucose after 1 hour: 185 mg/dL,” “Activity: exercising, predicted decrease in blood glucose after exercise: −18 mg/dL,” “Emotion: relaxation, predicted stability of blood glucose: 120 mg/dL.” These outputs are passed to subsequent notification units or insulin administration control units and used for threshold judgment and branching processing (e.g., high-risk notification during stress posts, specialized notification after exercise posts). The AI model is trained on GPU clusters using supervised learning (e.g., datasets labeled with post content, emotion, activity, and blood glucose fluctuation) and transfer learning (e.g., pre-training on public SNS datasets). Unlike conventional human judgment of post content or simple collection of all data, the analysis unit automatically performs integrated analysis of natural language, images, and time-series data, nonlinear priority inference, and rule-based prediction control using AI, greatly improving the efficiency of important data acquisition, anomaly detection accuracy, and the balance of user burden. Technical effects include (1) improved anomaly detection accuracy by utilizing the relationship between SNS activity and health status, (2) reduced storage and communication load by reducing unnecessary data, (3) reduced user burden. Specific application fields include stress management for young people, activity monitoring for athletes, home medical support, and health management in nursing care facilities. Furthermore, by linking multiple AI models (e.g., emotion estimation model, activity estimation model, vital priority estimation model), more advanced personalized prediction can be realized.

The notification unit can estimate the user's emotion and adjust the timing of notification based on the estimated emotion of the user. The notification unit can use AI, for example, to estimate the user's emotion. For example, the notification unit can estimate the user's emotion using facial recognition technology. The notification unit can also estimate the user's emotion using voice analysis technology. For example, the notification unit can analyze the tone and speed of the user's voice to estimate emotion. Furthermore, the notification unit can adjust the timing of notification based on the user's emotion. For example, when the user is feeling stressed, the notification unit can reduce the frequency of notifications to lessen the burden. When the user is relaxed, the notification unit can increase the frequency of notifications to provide more detailed information. Furthermore, when the user is exercising, the notification unit can notify after exercise. By adjusting the timing of notification according to the user's emotion, the user's burden can be reduced. Emotion estimation is realized using emotion engines or generative AI, for example, with emotion estimation functions. Generative AI may include text generation AI (e.g., LLM) or multimodal generative AI, but is not limited thereto. Specifically, the notification unit inputs the user's facial images (e.g., face image tensor shape=[3, 224, 224]), voice waveforms (e.g., one-dimensional array, sampling rate 16 kHz), and text data (e.g., speech recognition results or user input) into the AI model. The notification unit uses convolutional neural networks to extract feature maps from facial images and recurrent neural networks or Transformer-based models to estimate time-series emotional changes from voice waveforms and text data. Examples of input to the AI include (1) facial images taken during meetings, (2) voice data uttered during exercise, (3) text such as “I want to concentrate now.” Examples of AI model output include “Emotion label: stress, score: 0.82,” “Emotion label: relaxation, score: 0.15,” “Emotion label: concentration, score: 0.65,” and so on. The notification unit dynamically adjusts the notification timing control logic based on these emotion estimation results. For example, when the stress score is high, notification frequency is reduced to once per hour; when relaxed, detailed notifications are provided every 15 minutes; during exercise, exercise status is detected from accelerometer or heart rate changes, and notification is controlled to occur within 10 minutes after exercise. The AI model is trained on GPU clusters using supervised learning (e.g., notification history datasets labeled with emotion) and reinforcement learning (e.g., notification timing optimization with user satisfaction as reward). These notification timing control results are reflected in real time on user terminals and medical professional terminals, realizing optimal information transmission according to the user's mental and physical state. Unlike conventional human emotion judgment or fixed notification schedules, the notification unit automatically performs integrated analysis of high-dimensional multimodal data, nonlinear timing optimization, and situation-dependent notification control using AI, greatly improving usability, information transmission efficiency, and satisfaction. Technical effects include (1) reduced user burden by optimizing notification frequency and timing according to emotional state, (2) prevention of information transmission delay during important events, (3) provision of personalized notifications for each user, (4) reduced burden on healthcare professionals and family members. Specific application fields include home blood glucose management for diabetic patients, stress management support, health monitoring for athletes, and health management in nursing care facilities. Furthermore, by linking multiple emotion estimation models (e.g., CNN for facial expressions, RNN for voice, Transformer for text) and notification timing optimization models, more advanced personalized notification control can be realized.

The notification unit can refer to the user's past response history at the time of notification and select an optimal notification method. The notification unit can use AI, for example, to refer to the user's past response history. For example, the notification unit can preferentially select notification methods (voice, text, etc.) that the user has preferred in the past. The notification unit can also select a method for notifying at specific times based on the user's past response history. Furthermore, the notification unit can analyze the user's past response history and select the optimal notification device. By referring to the user's past response history, the optimal notification method can be selected. Specifically, the notification unit obtains notification history data recorded for each user (e.g., notification type, notification device ID, notification time, user response content, response speed, notification content type, etc., as structured data) from the cloud database and inputs it into the AI model. The notification unit uses recurrent neural networks or Transformer-based time-series pattern extraction models to analyze notification history data for the past month. Examples of input to the AI include (1) immediate response to voice notifications in the morning on weekdays, response to text notifications at night, (2) higher response rate to smartwatch notifications than smartphone notifications, (3) always immediate response to specific notification content (e.g., insulin administration instructions). The AI model automatically infers optimization of notification methods (e.g., automatic selection of voice, text, vibration, etc., for each time period), prioritization of notification devices (e.g., smartwatch when out, smart speaker when at home), and personalization of notification content (e.g., switching between detailed and summary notifications). Examples of AI model output include “Text notification recommended after 18:00 on weekdays,” “Vibration notification prioritized during exercise,” “Use both voice notification and smartwatch notification for insulin administration instructions.” These outputs are input to the control logic of the notification unit and reflected in the actual notification scheduler and device selection module. The AI model is trained on GPU clusters using supervised learning (e.g., notification history and user response labeled datasets) and reinforcement learning (e.g., optimization of response rate for each notification strategy). Unlike conventional human notification method setting or simple fixed notifications, the notification unit automatically performs pattern analysis of high-dimensional time-series history, simultaneous optimization of multiple factors, and nonlinear notification strategy inference using AI models, greatly improving notification efficiency, user satisfaction, and information transmission accuracy. Technical effects include (1) prevention of missed important notifications by improving response rate for each user, (2) reduced stress and burden by reducing unnecessary notifications, (3) improved operational efficiency by automating device selection. Specific application fields include home blood glucose management for diabetic patients, remote medical support, health management in nursing care facilities, and health monitoring for athletes. Furthermore, by linking multiple AI models (e.g., notification method optimization model and device selection model), more advanced personalized notification strategies can be realized.

The notification unit can customize notification content based on the user's current activity status or environment at the time of notification. The notification unit can use AI, for example, to grasp the user's current activity status or environment. For example, the notification unit can grasp the user's activity status based on exercise amount or heart rate. The notification unit can also grasp the user's environment based on temperature or humidity. Furthermore, the notification unit can customize notification content based on the user's current activity status or environment. For example, when the user is exercising, the notification unit can customize notification content after exercise. When the user is sleeping, the notification unit can customize notification content after waking up. Furthermore, when the user is out, the notification unit can customize notification content at the location outside. By customizing notification content based on the user's activity status or environment, more appropriate notifications can be provided. Specifically, the notification unit simultaneously obtains multidimensional vital data (e.g., time-series tensor with shape=[number of sensor types, number of samples]) from accelerometer, heart rate sensor, skin temperature sensor, etc., and data from environmental sensors (e.g., temperature, humidity, illuminance, atmospheric pressure), and inputs them into the AI model. The notification unit uses convolutional neural networks, recurrent neural networks, or Transformer-based time-series classification models to estimate the user's activity state (e.g., resting, exercising, sleeping, going out) and environmental state (e.g., indoor, outdoor, high temperature, low humidity). Examples of input to the AI include (1) exercise data with large fluctuations in accelerometer values and increased heart rate, (2) sleep data with stable heart rate and body temperature and low illuminance, (3) outdoor activity data with high temperature and humidity. Examples of AI model output include “Activity state: exercising,” “Environmental state: outdoor, high temperature,” “Activity state: sleeping,” and so on. The notification unit automatically applies customization rules for notification content based on these estimation results (e.g., during exercise, send summary notification of data for 10 minutes after exercise; during sleep, send summary notification after waking up; during outings, send only key points). The AI model is trained on GPU clusters using supervised learning (e.g., notification history datasets labeled with activity and environment) to minimize the loss function (e.g., cross-entropy). Unlike conventional human activity/environment judgment or simple sending of all notifications, the notification unit automatically performs multivariate analysis of high-dimensional sensor data, nonlinear state estimation, and rule-based notification content generation using AI models, greatly improving the relevance, usefulness, and efficiency of notifications. Technical effects include (1) reduced storage and communication load by reducing unnecessary notifications, (2) improved anomaly detection rate by high-precision notifications during important events, (3) reduced user burden. Application fields include home monitoring for diabetic patients, training management for athletes, health management in nursing care facilities, and remote medical support. Furthermore, by linking multiple AI models (e.g., activity estimation model, environment estimation model, notification content generation model), more advanced personalized notifications can be realized.

The notification unit can estimate the user's emotion and determine the priority of notification based on the estimated emotion of the user. The notification unit can use AI, for example, to estimate the user's emotion. For example, the notification unit can estimate the user's emotion using facial recognition technology. The notification unit can also estimate the user's emotion using voice analysis technology. For example, the notification unit can analyze the tone and speed of the user's voice to estimate emotion. Furthermore, the notification unit can determine the priority of notification based on the user's emotion. For example, when the user is feeling stressed, the notification unit can prioritize only important notifications. When the user is relaxed, the notification unit can prioritize detailed notifications. Furthermore, when the user is exercising, the notification unit can prioritize important notifications after exercise. By determining the priority of notification according to the user's emotion, important notifications can be prioritized. Emotion estimation is realized using emotion engines or generative AI, for example, with emotion estimation functions. Generative AI may include text generation AI (e.g., LLM) or multimodal generative AI, but is not limited thereto. Specifically, the notification unit inputs the user's facial images (e.g., face image tensor shape=[3, 224, 224]), voice waveforms (e.g., one-dimensional array, sampling rate 16 kHz), and text data (e.g., speech recognition results or user input) into the AI model, which outputs emotion labels (e.g., “stress,” “relaxation,” “tension,” etc.) and emotion scores (e.g., continuous values from 0.0 to 1.0). Examples of input to the AI include (1) facial images during meetings, (2) voice data during exercise, (3) text input into the application. Examples of AI model output include “Emotion label: stress, score: 0.82,” “Emotion label: relaxation, score: 0.15,” and so on. The notification unit automatically determines notification priority control logic based on these emotion estimation results (e.g., prioritize emergency notifications during stress, detailed notifications during relaxation, important notifications after exercise). The AI model is trained on GPU clusters using supervised learning (e.g., notification history datasets labeled with emotion) and reinforcement learning (e.g., optimization of user satisfaction for each notification priority). Unlike conventional human emotion judgment or simple sending of all notifications, the notification unit automatically performs integrated analysis of high-dimensional multimodal data, nonlinear priority inference, and rule-based notification control using AI models, greatly improving the efficiency of important notification acquisition, anomaly detection accuracy, and the balance of user burden. Technical effects include (1) improved anomaly detection rate by high-precision notifications during important events, (2) reduced storage and communication load by reducing unnecessary notifications, (3) reduced user burden. Application fields include home monitoring for diabetic patients, stress management support, condition management for athletes, and health monitoring in nursing care settings. Furthermore, by combining multiple emotion estimation models (e.g., CNN for facial expressions, RNN for voice, Transformer for text), notification priority estimation accuracy and optimization of notification control can be further improved.

The notification unit can select an optimal notification method by taking into account the user's geographic location information at the time of notification. For example, the notification unit can utilize AI to acquire the user's geographic location information. The notification unit can obtain the user's location information based on GPS data, or acquire geographic location information by using location information services. Furthermore, the notification unit can select the optimal notification method based on the user's geographic location information. For instance, if the user is in a high-altitude area, the notification unit can customize the notification content based on oxygen saturation data. If the user is in an urban area, the notification unit can customize the notification content based on heart rate and blood pressure data. If the user is at home, the notification unit can customize the notification content based on body temperature and blood glucose level data. By considering the user's geographic location information, the optimal notification method can be selected. Specifically, the notification unit acquires location information (e.g., latitude, longitude, altitude, location accuracy, timestamp, etc. as structured data) in real time from GPS sensors or Wi-Fi/Bluetooth beacons, and integrates it with vital data (time-series tensors of heart rate, blood pressure, body temperature, and blood glucose levels) as input to an AI model. The notification unit uses a Transformer-based spatiotemporal multivariate analysis model to automatically learn the correlation and environmental dependencies between location information and vital data, and optimizes notification content and methods. Examples of AI input include (1) vital data at high altitude (elevation 2000 m), (2) data in urban areas (with latitude/longitude, elevation, temperature, PM2.5 concentration, etc.), and (3) data at home (determined by Wi-Fi beacon ID). Examples of AI model output include: “Current location: high altitude, recommend voice notification+vibration notification when oxygen saturation decreases”; “Current location: urban area, prioritize text notification when heart rate or blood pressure fluctuates”; “Current location: home, provide detailed notification when body temperature and blood glucose levels are stable.” The notification unit automatically controls the notification method (e.g., voice, text, vibration, push notification, etc.) and customizes notification content based on these outputs. The AI model is trained on a GPU cluster using supervised learning (e.g., location-labeled notification history datasets) and reinforcement learning (e.g., optimization of notification strategies for each location). Unlike conventional human-based location determination or simple mass notification, the notification unit automatically performs multivariate analysis of high-dimensional spatiotemporal data, nonlinear notification method inference, and rule-based notification control using AI, resulting in significant improvements in relevant notification acquisition, anomaly detection accuracy, and operational efficiency. Technical effects include: (1) early detection and notification of health risks with high location dependency; (2) reduction of storage and communication load by eliminating unnecessary notifications; (3) reduction of user burden; and so on. Specific application fields include health management for mountaineers and high-altitude residents, lifestyle disease monitoring in urban areas, home medical support, and health management in nursing care facilities. Furthermore, by linking multiple AI models (e.g., location estimation model+notification method optimization model), more advanced personalized notification strategies can be realized.

The notification unit can analyze the user's social media activity at the time of notification and notify relevant information. For example, the notification unit can utilize AI to analyze the user's social media activity. If the user posts about feeling stressed, the notification unit can customize the notification content based on heart rate and blood pressure data. If the user posts about being relaxed, the notification unit can customize the notification content based on body temperature and blood glucose level data. If the user posts about exercising, the notification unit can customize the notification content based on post-exercise heart rate and blood glucose level data. By analyzing the user's social media activity, the notification unit can notify relevant information. Specifically, the notification unit acquires the user's public social media posts (e.g., text, images, videos, posting time, location information, etc. as structured data) via API and inputs them into a natural language processing model (e.g., large language model or multimodal analysis model). The notification unit estimates the user's emotional state (e.g., stress, relaxation, exercising, etc.) and activity status from the post content, integrates it with vital data (time-series tensors of heart rate, blood pressure, body temperature, and blood glucose levels), and inputs it into a notification content generation model. Examples of AI input include (1) stress posts such as “Today was a tough day at work” plus heart rate and blood pressure data from the past hour; (2) exercise posts such as “Jogging in the park” plus post-exercise vital data; (3) relaxation posts such as “Spending a relaxing holiday” plus body temperature and blood glucose level data. Examples of AI model output include: “Emotion: stress, key point notification+emergency notification when heart rate or blood pressure is abnormal”; “Activity: exercising, notify blood glucose level changes after exercise”; “Emotion: relaxation, provide detailed notification.” The notification unit automatically controls notification content, frequency, and method based on these outputs. The AI model is trained on a GPU cluster using supervised learning (e.g., datasets labeled with post content, emotion, activity, and notification content) and transfer learning (e.g., pre-training on public SNS datasets). Unlike conventional human-based post content determination or simple mass notification, the notification unit automatically performs integrated analysis of natural language, images, and time-series data, nonlinear notification content inference, and rule-based notification control using AI, resulting in significant improvements in important notification acquisition efficiency, anomaly detection accuracy, and user burden balance. Technical effects include: (1) improved anomaly detection accuracy by leveraging the relationship between SNS activity and health status; (2) reduction of storage and communication load by eliminating unnecessary notifications; (3) reduction of user burden; and so on. Application fields include stress management for young people, activity monitoring for athletes, home medical support, and health management in nursing care facilities. Furthermore, by linking multiple AI models (e.g., emotion estimation model+activity estimation model+notification content generation model), more advanced personalized notifications can be realized.

The suggestion unit can estimate the user's emotion and adjust the meal suggestion method based on the estimated emotion of the user. For example, the suggestion unit can utilize AI to estimate the user's emotion. The suggestion unit can estimate the user's emotion using facial recognition technology, or by analyzing voice data. For instance, the suggestion unit can analyze the tone and speed of the user's voice to estimate emotion. Furthermore, the suggestion unit can adjust the meal suggestion method based on the user's emotion. For example, if the user is feeling stressed, the suggestion unit can propose ingredients with relaxing effects. If the user is relaxed, the suggestion unit can propose nutritionally balanced meals. If the user has just exercised, the suggestion unit can propose ingredients suitable for energy replenishment. By adjusting the meal suggestion method according to the user's emotion, more appropriate meal suggestions can be provided. Emotion estimation is realized using, for example, an emotion engine or generative AI with emotion estimation functions. Generative AI may include text generation AI (e.g., LLM) or multimodal generative AI, but is not limited to these examples. Specifically, the suggestion unit inputs the user's facial images (e.g., face image tensor shape=[3, 224, 224]), voice waveforms (e.g., 1D array, sampling rate 16 kHz), and text data (e.g., speech recognition results or user input) into an AI model. The suggestion unit uses convolutional neural networks to extract feature maps from facial images, and recurrent neural networks or Transformer-based models to estimate time-series emotional changes from voice waveforms and text data. Examples of AI input include (1) facial images taken during a meeting, (2) voice data spoken during exercise, and (3) text such as “I'm tired today.” Examples of AI model output include “Emotion label: stress, score: 0.82” and “Emotion label: relaxation, score: 0.15.” The suggestion unit dynamically adjusts the parameters and input weighting of the meal suggestion generation model (e.g., large language model or multimodal generation model) based on these emotion estimation results. For example, when the stress score is high, ingredients with relaxing effects (e.g., herbal tea, seafood, foods rich in vitamin B group) and simple cooking methods are prioritized; when relaxed, new recipes and diverse suggestions are provided; after exercise, high-protein and high-carbohydrate energy replenishment ingredients (e.g., chicken breast, banana, brown rice) are recommended. Examples of AI model output include “For dinner tonight, we recommend a low-carb salad with chicken breast and broccoli,” “To relieve stress, we suggest mackerel dishes containing omega-3 fatty acids,” and “Consume banana and yogurt within 30 minutes after exercise.” These outputs are displayed in natural language on the user terminal UI, providing optimal meal suggestions in real time according to the user's condition. The AI model is trained on a GPU cluster using supervised learning (e.g., emotion-labeled meal history and blood glucose variation datasets) and reinforcement learning (e.g., optimization with blood glucose stability after meal suggestion as reward). Unlike conventional human-based emotion determination or simple recipe recommendation, the suggestion unit automatically performs integrated analysis of high-dimensional multimodal data, nonlinear suggestion generation, and context-dependent model switching using AI, resulting in significant improvements in meal suggestion accuracy, speed, and personalization. Technical effects include: (1) optimization of meal suggestion accuracy according to emotional state to suppress blood glucose fluctuations; (2) improvement of user satisfaction; (3) reduction of burden on medical professionals and family; (4) automation of meal management; and so on. Specific application fields include home meal management for diabetic patients, stress management support, nutrition management for athletes, and meal suggestions in nursing care facilities. Furthermore, by linking multiple emotion estimation models (e.g., CNN for facial images, RNN for voice, Transformer for text) and meal suggestion generation models, more advanced personalized meal suggestions can be realized.

The suggestion unit can refer to the user's past meal history at the time of suggestion to provide optimal suggestions. For example, the suggestion unit can utilize AI to refer to the user's past meal history. The suggestion unit can propose optimal meals based on ingredients the user has preferred in the past, or suggest nutritionally balanced meals based on past meal history. Furthermore, the suggestion unit can analyze the user's past meal history to propose healthy meals. By referring to the user's past meal history, optimal meal suggestions can be provided. Specifically, the suggestion unit inputs meal history data accumulated in a cloud database for each user (e.g., ingredient ID, recipe ID, intake time, nutrient amounts, blood glucose variation as time-series arrays) into an AI model. The suggestion unit uses recurrent neural networks or Transformer-based time-series pattern extraction models to analyze meal history data for the past month. Examples of AI input include (1) history of consuming bread and eggs for breakfast every day, (2) history of frequent fish dishes on weekends, and (3) history of rapid blood glucose increase after consuming specific ingredients. The AI model automatically extracts the user's preference patterns (e.g., preference for Japanese, Western, or Chinese cuisine), nutritional balance (e.g., protein, fat, carbohydrate ratios), and correlation with blood glucose variation, and generates optimal meal suggestions. Examples of AI model output include “Vegetable intake has been insufficient over the past week, so a vegetable-focused menu is suggested for tonight,” “Fish dishes are associated with stable blood glucose, so fish dishes are recommended twice a week,” and “On days with high bread intake at breakfast, blood glucose tends to rise, so switching to low-carb bread is suggested.” These outputs are displayed in natural language on the user terminal UI, providing personalized suggestions in real time based on the user's past meal history. The AI model is trained on a GPU cluster using supervised learning (e.g., meal history, blood glucose variation, and preference-labeled datasets) and reinforcement learning (e.g., optimization with blood glucose stability after suggestion as reward). Unlike conventional human-based meal history reference or simple recipe recommendation, the suggestion unit automatically performs multivariate analysis of high-dimensional time-series data, nonlinear pattern extraction, and personalized suggestion generation using AI, resulting in significant improvements in meal suggestion accuracy, speed, and user satisfaction. Technical effects include: (1) improved meal suggestion accuracy based on past history; (2) suppression of blood glucose variation; (3) improvement of user satisfaction; (4) reduction of burden on medical professionals and family; and so on. Specific application fields include home meal management for diabetic patients, health management support, nutrition management for athletes, and meal suggestions in nursing care facilities. Furthermore, by linking multiple history analysis models (e.g., preference extraction model+blood glucose variation prediction model), more advanced personalized meal suggestions can be realized.

The suggestion unit can customize meal suggestions based on the user's current health status and lifestyle at the time of suggestion. For example, the suggestion unit can utilize AI to understand the user's current health status and lifestyle. The suggestion unit can determine the user's current health status based on body weight and blood pressure, or understand lifestyle habits based on exercise habits and sleep patterns. Furthermore, the suggestion unit can customize meal suggestions based on the user's current health status and lifestyle. For example, if the user is tired, the suggestion unit can propose ingredients suitable for energy replenishment. If the user has healthy lifestyle habits, the suggestion unit can propose nutritionally balanced meals. If the user is feeling unwell, the suggestion unit can propose easily digestible ingredients. By customizing meal suggestions according to health status and lifestyle, more appropriate meal suggestions can be provided. Specifically, the suggestion unit acquires the user's latest vital data (e.g., body weight, blood pressure, heart rate, blood glucose level as numerical vectors), exercise history (e.g., exercise type, intensity, duration, calories burned), sleep history (e.g., bedtime, wake-up time, sleep depth, sleep efficiency), and lifestyle questionnaire data (e.g., frequency of drinking, smoking, snacking) from a cloud database and inputs them into an AI model. The suggestion unit uses Transformer-based multivariate time-series analysis models or multimodal integration models to automatically learn the complex causal relationships among health status, lifestyle, and vital data, and generates optimal meal suggestions. Examples of AI input include (1) time-series data of blood pressure, body weight, sleep time, and exercise amount for the past week; (2) subjective health questionnaires such as “I feel tired recently”; (3) exercise frequency, intensity, and sleep efficiency for the past month. Examples of AI model output include “To recover from fatigue, pork dishes rich in vitamin B group are suggested,” “For health maintenance, a vegetable-focused balanced menu is recommended,” and “When feeling unwell, rice porridge or soup that is easy to digest is recommended.” These outputs are displayed in natural language on the user terminal UI, providing personalized meal suggestions in real time according to the user's health status and lifestyle. The AI model is trained on a GPU cluster using supervised learning (e.g., datasets labeled with health status, lifestyle, meal history, and blood glucose variation) and reinforcement learning (e.g., optimization with improvement in health indicators after meal suggestion as reward). Unlike conventional human-based health status determination or simple recipe recommendation, the suggestion unit automatically performs integrated analysis of high-dimensional multivariate data, nonlinear causal inference, and personalized suggestion generation using AI, resulting in significant improvements in meal suggestion accuracy, speed, and user satisfaction. Technical effects include: (1) improved meal suggestion accuracy by considering health status and lifestyle factors; (2) suppression of blood glucose variation; (3) improvement of user satisfaction; (4) reduction of burden on medical professionals and family; and so on. Specific application fields include home meal management for diabetic patients, health management support, nutrition management for athletes, and meal suggestions in nursing care facilities. Furthermore, by linking multiple lifestyle analysis models (e.g., RNN for exercise history, Transformer for sleep history, vital data integration model), more advanced personalized meal suggestions can be realized.

The suggestion unit can estimate the user's emotion and determine the priority of meal suggestions based on the estimated emotion of the user. For example, the suggestion unit can utilize AI to estimate the user's emotion. The suggestion unit can estimate the user's emotion using facial recognition technology, or by analyzing voice data. For instance, the suggestion unit can analyze the tone and speed of the user's voice to estimate emotion. Furthermore, the suggestion unit can determine the priority of meal suggestions based on the user's emotion. For example, if the user is feeling stressed, the suggestion unit can prioritize ingredients with relaxing effects. If the user is relaxed, the suggestion unit can prioritize nutritionally balanced meals. If the user has just exercised, the suggestion unit can prioritize ingredients suitable for energy replenishment. By determining the priority of meal suggestions according to the user's emotion, more appropriate meal suggestions can be provided. Emotion estimation is realized using, for example, an emotion engine or generative AI with emotion estimation functions. Generative AI may include text generation AI (e.g., LLM) or multimodal generative AI, but is not limited to these examples. Specifically, the suggestion unit inputs the user's facial images (e.g., face image tensor shape=[3,224,224]), voice waveforms (e.g., 1D array, sampling rate 16 kHz), text data (e.g., speech recognition results or user input), etc. into an AI model, which outputs emotion labels (e.g., “stress,” “relaxation,” “tension,” etc.) and emotion scores (e.g., continuous values from 0.0 to 1.0). Examples of AI input include (1) facial images during a meeting, (2) voice data during exercise, and (3) app input text. Examples of AI model output include “Emotion label: stress, score: 0.82” and “Emotion label: relaxation, score: 0.15.” The suggestion unit automatically determines the priority of the meal suggestion list based on these emotion estimation results (e.g., prioritize ingredients with high relaxing effects during stress, focus on nutritional balance during relaxation, focus on energy replenishment after exercise). The AI model is trained on a GPU cluster using supervised learning (e.g., emotion-labeled meal history and blood glucose variation datasets) and reinforcement learning (e.g., optimization with blood glucose stability and user satisfaction after suggestion as reward). Unlike conventional human-based emotion determination or simple mass suggestion, the suggestion unit automatically performs integrated analysis of high-dimensional multimodal data, nonlinear priority inference, and rule-based suggestion control using AI, resulting in significant improvements in important suggestion acquisition efficiency, anomaly detection accuracy, and user satisfaction balance. Technical effects include: (1) suppression of blood glucose variation by high-accuracy suggestions during important events; (2) reduction of unnecessary suggestions to reduce user burden; (3) improvement of user satisfaction; and so on. Application fields include home meal management for diabetic patients, stress management support, nutrition management for athletes, and meal suggestions in nursing care settings. Furthermore, by combining multiple emotion estimation models (e.g., CNN for facial images, RNN for voice, Transformer for text) and suggestion priority estimation models, suggestion accuracy and control optimization can be further enhanced.

The suggestion unit can provide optimal meal suggestions by taking into account the user's geographic location information at the time of suggestion. For example, the suggestion unit can utilize AI to acquire the user's geographic location information. The suggestion unit can obtain the user's location information based on GPS data, or acquire geographic location information by using location information services. Furthermore, the suggestion unit can provide optimal meal suggestions based on the user's geographic location information. For instance, if the user is in a high-altitude area, the suggestion unit can customize meal suggestions based on oxygen saturation data. If the user is in an urban area, the suggestion unit can customize meal suggestions based on heart rate and blood pressure data. If the user is at home, the suggestion unit can customize meal suggestions based on body temperature and blood glucose level data. By considering the user's geographic location information, optimal meal suggestions can be provided. Specifically, the suggestion unit acquires location information (e.g., latitude, longitude, altitude, location accuracy, timestamp, etc. as structured data) in real time from GPS sensors or Wi-Fi/Bluetooth beacons, and integrates it with vital data (time-series tensors of heart rate, blood pressure, body temperature, and blood glucose levels) as input to an AI model. The suggestion unit uses a Transformer-based spatiotemporal multivariate analysis model to automatically learn the correlation and environmental dependencies between location information and vital data, and optimizes meal suggestion content and methods. Examples of AI input include (1) vital data at high altitude (elevation 2000 m), (2) data in urban areas (with latitude/longitude, elevation, temperature, PM2.5 concentration, etc.), and (3) data at home (determined by Wi-Fi beacon ID). Examples of AI model output include: “Current location: high altitude, suggest ingredients rich in iron and vitamin C when oxygen saturation decreases”; “Current location: urban area, recommend low-salt and potassium-rich meals when heart rate or blood pressure fluctuates”; “Current location: home, suggest a normal balanced menu when body temperature and blood glucose levels are stable.” These outputs are displayed in natural language on the user terminal UI, providing personalized meal suggestions in real time according to the user's geographic location information. The AI model is trained on a GPU cluster using supervised learning (e.g., location-labeled vital and meal history datasets) and reinforcement learning (e.g., optimization of meal suggestions for each location). Unlike conventional human-based location determination or simple mass suggestion, the suggestion unit automatically performs multivariate analysis of high-dimensional spatiotemporal data, nonlinear suggestion content inference, and rule-based suggestion control using AI, resulting in significant improvements in relevant suggestion acquisition, anomaly detection accuracy, and operational efficiency. Technical effects include: (1) early detection of health risks with high location dependency and meal suggestions; (2) reduction of unnecessary suggestions to reduce user burden; (3) improvement of user satisfaction; and so on. Specific application fields include health management for mountaineers and high-altitude residents, lifestyle disease monitoring in urban areas, home medical support, and meal suggestions in nursing care facilities. Furthermore, by linking multiple AI models (e.g., location estimation model+meal suggestion optimization model), more advanced personalized meal suggestions can be realized.

The suggestion unit can analyze the user's social media activity at the time of suggestion and provide relevant meal suggestions. For example, the suggestion unit can utilize AI to analyze the user's social media activity. If the user posts about feeling stressed, the suggestion unit can propose ingredients with relaxing effects. If the user posts about being relaxed, the suggestion unit can propose nutritionally balanced meals. If the user posts about exercising, the suggestion unit can propose ingredients suitable for energy replenishment. By analyzing the user's social media activity, relevant meal suggestions can be provided. Specifically, the suggestion unit acquires the user's public social media posts (e.g., text, images, videos, posting time, location information, etc. as structured data) via API and inputs them into a natural language processing model (e.g., large language model or multimodal analysis model). The suggestion unit estimates the user's emotional state (e.g., stress, relaxation, exercising, etc.) and activity status from the post content, integrates it with vital data (time-series tensors of heart rate, blood pressure, body temperature, and blood glucose levels), and inputs it into a meal suggestion generation model. Examples of AI input include (1) stress posts such as “Today was a tough day at work” plus heart rate and blood pressure data from the past hour; (2) exercise posts such as “Jogging in the park” plus post-exercise vital data; (3) relaxation posts such as “Spending a relaxing holiday” plus body temperature and blood glucose level data. Examples of AI model output include: “Emotion: stress, suggest herbal tea or fish dishes with relaxing effects”; “Activity: exercising, recommend banana and yogurt for energy replenishment after exercise”; “Emotion: relaxation, suggest a vegetable-focused balanced menu.” These outputs are displayed in natural language on the user terminal UI, providing personalized meal suggestions in real time according to the user's SNS activity. The AI model is trained on a GPU cluster using supervised learning (e.g., datasets labeled with post content, emotion, activity, and meal suggestion) and transfer learning (e.g., pre-training on public SNS datasets). Unlike conventional human-based post content determination or simple mass suggestion, the suggestion unit automatically performs integrated analysis of natural language, images, and time-series data, nonlinear suggestion content inference, and rule-based suggestion control using AI, resulting in significant improvements in important suggestion acquisition efficiency, anomaly detection accuracy, and user satisfaction balance. Technical effects include: (1) improved meal suggestion accuracy by leveraging the relationship between SNS activity and health status; (2) reduction of unnecessary suggestions to reduce user burden; (3) improvement of user satisfaction; and so on. Application fields include stress management for young people, activity monitoring for athletes, home medical support, and meal suggestions in nursing care facilities. Furthermore, by linking multiple AI models (e.g., emotion estimation model +activity estimation model +meal suggestion generation model), more advanced personalized meal suggestions can be realized.

The system according to the embodiment is not limited to the examples described above, and various modifications are possible, for example, as follows. Specifically, the system allows for diverse variations in AI model architecture and training methods, data flow, sensor configuration, notification and suggestion means, and so on. For example, as AI models, convolutional neural networks, recurrent neural networks, Transformer-based models, graph neural networks, self-supervised learning models, and reinforcement learning models can be used in combination. As data input, vital data (heart rate, blood pressure, body temperature, blood glucose level, etc.), environmental data (temperature, humidity, illuminance, etc.), location information (GPS, Wi-Fi beacon, etc.), voice, image, text, video data, social media posts, lifestyle questionnaires, etc. can be integrated in a multivariate and multimodal manner. Notification and suggestion means may include smartphones, smartwatches, smart speakers, tablets, PCs, electronic paper devices, speech synthesis devices, vibration devices, and so on. As AI model training methods, supervised learning, reinforcement learning, transfer learning, self-supervised learning, and federated learning can be applied. Furthermore, by linking multiple AI models, each function such as emotion estimation, activity estimation, vital prediction, meal suggestion, and notification optimization can be modularized and distributed via APIs or message queues. As a result, the system can be flexibly configured and expanded according to the needs and usage environment of each user. Technical effects include: (1) improved scalability and maintainability of the system; (2) rapid introduction of new sensors and AI algorithms; (3) improved adaptability to diverse use cases; (4) advancement of personalized health support; and so on. Specific application fields include home blood glucose management for diabetic patients, remote medical support, health monitoring in nursing care facilities, health management for athletes, corporate health management support, stress management, nutrition guidance, rehabilitation support, and lifestyle disease prevention.

The analysis unit can analyze the user's sleep patterns and predict changes in blood glucose levels based on sleep quality. For example, the analysis unit can analyze the user's sleep duration and sleep depth to predict the impact of sleep deprivation on blood glucose levels. The analysis unit can also analyze changes in heart rate and body temperature during sleep and predict changes in blood glucose levels based on these data. Furthermore, the analysis unit can predict changes in blood glucose levels by considering the user's sleep environment (e.g., room temperature and humidity). By considering the user's sleep patterns and environment, more accurate prediction of changes in blood glucose levels is possible. Specifically, the analysis unit acquires multidimensional time-series data constituting the user's sleep patterns (e.g., sleep start/end time, ratio of deep/light sleep, minute-by-minute arrays of heart rate, body temperature, respiration during sleep, shape=[6,480], etc.) and sleep environment data (e.g., time-series vectors of room temperature, humidity, illuminance, noise level) from a cloud database and inputs them into an AI model. The analysis unit uses convolutional neural networks to extract feature maps from biosignals during sleep, and recurrent neural networks or Transformer-based time-series analysis models to learn the long-term effects of sleep patterns and environmental changes. Examples of AI input include (1) time-series data of sleep depth, heart rate, and body temperature for the past 7 days; (2) transitions of room temperature, humidity, and noise level during sleep; (3) vital variation data for days with sleep deprivation and days with sufficient sleep. Examples of AI model output include “Predicted blood glucose increase after sleep deprivation: +15 mg/dL,” “Blood glucose variation risk score when deep sleep ratio is below 40%: 0.72,” and “Prediction of blood glucose instability when room temperature exceeds 28°C.” The analysis unit dynamically adjusts the parameters of the blood glucose variation prediction model (e.g., emphasize rapid variation during sleep deprivation, strengthen smoothing parameters during deep sleep) based on these outputs to optimize prediction accuracy. These prediction results are passed to subsequent notification units or insulin administration control units and used for threshold determination and branching processing (e.g., emergency notification in case of high risk, regular notification in normal cases). The AI model is trained on a parallel computing cluster using GPUs with supervised learning (e.g., datasets labeled with sleep patterns, environment, and blood glucose variation) and self-supervised learning (e.g., pre-training of features by time-series prediction tasks on sleep data). Unlike conventional human-based sleep record reference or simple rule-based prediction, the analysis unit automatically performs integrated analysis of high-dimensional multimodal data, nonlinear causal inference, and context-dependent model switching using AI, resulting in significant improvements in prediction accuracy, speed, and reproducibility of blood glucose variation. Technical effects include: (1) reduction of prediction error by considering sleep patterns and environmental factors; (2) early detection of abnormal events; (3) personalized prediction for each user; (4) reduction of burden on medical professionals and family; and so on. Specific application fields include home blood glucose management for diabetic patients, health monitoring for patients with sleep disorders, condition management for athletes, and health management in nursing care facilities. Furthermore, by linking multiple sleep analysis models (e.g., sleep depth estimation model+environmental impact estimation model+blood glucose variation prediction model), more advanced personalized health support can be realized.

The notification unit can analyze the user's exercise history and notify the timing of insulin injection based on changes in blood glucose levels after exercise. For example, the notification unit can analyze the user's exercise intensity and duration to predict changes in blood glucose levels after exercise. The notification unit can also analyze changes in heart rate and body temperature after exercise and notify the timing of insulin injection based on these data. Furthermore, the notification unit can notify the timing of insulin injection by considering the user's exercise environment (e.g., whether the exercise was outdoors or indoors). By considering the user's exercise history and environment, more appropriate notification of insulin injection timing can be provided. Specifically, the notification unit acquires the user's exercise history (e.g., exercise type, intensity, duration, calories burned, exercise start/end time as time-series arrays), post-exercise vital data (e.g., time-series tensors of heart rate, body temperature, blood glucose level, shape=[3,60]), and exercise environment data (e.g., outdoor/indoor determination, temperature, humidity, weather, etc.) from a cloud database and inputs them into an AI model. The notification unit uses convolutional neural networks to extract feature maps from biosignals after exercise, and recurrent neural networks or Transformer-based time-series analysis models to learn the complex effects of exercise history, environment, and vital variation. Examples of AI input include (1) heart rate, body temperature, and blood glucose data after 30 minutes of running (outdoors, temperature 25° C.); (2) vital data after 20 minutes of high-intensity indoor strength training; (3) blood glucose trends after 60 minutes of low-intensity walking. Examples of AI model output include “Predicted blood glucose decrease 30 minutes after exercise: −18 mg/dL, recommended insulin injection timing: 40 minutes after exercise,” “After high-intensity exercise, recommend delaying insulin injection by 1 hour,” and “For outdoor exercise, automatically adjust injection timing according to temperature and humidity.” The notification unit dynamically adjusts the control logic of the insulin injection notification scheduler based on these outputs and provides optimal notifications in real time to user terminals and medical professional terminals. The AI model is trained on a parallel computing cluster using GPUs with supervised learning (e.g., datasets labeled with exercise history, vital data, and injection timing) and reinforcement learning (e.g., optimization with blood glucose stability for each injection timing as reward). Unlike conventional human-based exercise record reference or simple fixed schedule notification, the notification unit automatically performs integrated analysis of high-dimensional multimodal data, nonlinear timing optimization, and context-dependent notification control using AI, resulting in significant improvements in the accuracy, usability, and safety of insulin injection timing. Technical effects include: (1) optimization of injection timing considering exercise history and environmental factors to suppress blood glucose variation; (2) reduction of hypoglycemia and hyperglycemia risk; (3) reduction of user burden; (4) reduction of burden on medical professionals and family; and so on. Specific application fields include home blood glucose management for diabetic patients, health management for athletes, insulin administration support in nursing care facilities, and remote medical support. Furthermore, by linking multiple exercise analysis models (e.g., exercise type estimation model+environmental impact estimation model+injection timing optimization model), more advanced personalized notifications can be realized.

The suggestion unit can analyze the user's meal history and provide meal suggestions based on past meal patterns. For example, the suggestion unit can analyze ingredients and recipes consumed by the user in the past to identify meals that affected changes in blood glucose levels. The suggestion unit can also predict the impact of specific ingredients or recipes on blood glucose levels based on meal history and provide meal suggestions accordingly. Furthermore, the suggestion unit can propose nutritionally balanced meals based on the user's meal history. By considering the user's past meal history, more appropriate meal suggestions can be provided. Specifically, the suggestion unit inputs meal history data accumulated in a cloud database for each user (e.g., ingredient ID, recipe ID, intake time, nutrient amounts, blood glucose variation as time-series arrays shape=[5,90], etc.) into an AI model. The suggestion unit uses recurrent neural networks or Transformer-based time-series pattern extraction models to analyze meal history data for the past month, automatically extracting the contribution of each ingredient and recipe to blood glucose variation and trends in nutritional balance. Examples of AI input include (1) history of consuming bread and eggs for breakfast every day, (2) history of frequent fish dishes on weekends, and (3) history of rapid blood glucose increase after consuming specific ingredients. The AI model automatically extracts the user's preference patterns (e.g., preference for Japanese, Western, or Chinese cuisine), nutritional balance (e.g., protein, fat, carbohydrate ratios), and correlation with blood glucose variation, and generates optimal meal suggestions. Examples of AI model output include “Vegetable intake has been insufficient over the past week, so a vegetable-focused menu is suggested for tonight,” “Fish dishes are associated with stable blood glucose, so fish dishes are recommended twice a week,” and “On days with high bread intake at breakfast, blood glucose tends to rise, so switching to low-carb bread is suggested.” These outputs are displayed in natural language on the user terminal UI, providing personalized suggestions in real time based on the user's past meal history. The AI model is trained on a parallel computing cluster using GPUs with supervised learning (e.g., meal history, blood glucose variation, and preference-labeled datasets) and reinforcement learning (e.g., optimization with blood glucose stability after suggestion as reward). Unlike conventional human-based meal history reference or simple recipe recommendation, the suggestion unit automatically performs multivariate analysis of high-dimensional time-series data, nonlinear pattern extraction, and personalized suggestion generation using AI, resulting in significant improvements in meal suggestion accuracy, speed, and user satisfaction. Technical effects include: (1) improved meal suggestion accuracy based on past history; (2) suppression of blood glucose variation; (3) improvement of user satisfaction; (4) reduction of burden on medical professionals and family; and so on. Specific application fields include home meal management for diabetic patients, health management support, nutrition management for athletes, and meal suggestions in nursing care facilities. Furthermore, by linking multiple history analysis models (e.g., preference extraction model+blood glucose variation prediction model), more advanced personalized meal suggestions can be realized.

The collection unit can analyze the user's stress level and adjust the timing of vital data collection by considering the impact of stress on blood glucose levels. For example, the collection unit can analyze changes in the user's heart rate and blood pressure to estimate stress level. If the user's stress level is high, the collection unit can increase the frequency of vital data collection to obtain more detailed data. If the user's stress level is low, the collection unit can reduce the collection frequency to lessen the user's burden. By considering the user's stress level, more appropriate vital data collection is possible. Specifically, the collection unit inputs vital data such as heart rate, blood pressure, skin conductance, respiration rate (e.g., time-series tensor shape=[4,60] at 1-minute intervals), and multimodal data such as facial images, voice waveforms, and text data into an AI model. The collection unit uses convolutional neural networks to extract feature maps from facial images, and recurrent neural networks or Transformer-based models to estimate stress level from voice, text, and vital time-series data. Examples of AI input include (1) heart rate, facial images, and voice data during a meeting; (2) blood pressure, respiration rate, and voice data during exercise; (3) text such as “I was busy and tired today.” Examples of AI model output include “Stress level: high, score: 0.85” and “Stress level: low, score: 0.12.” The collection unit automatically optimizes the timing and frequency control logic for vital data collection based on these stress estimation results. For example, when the stress score is high, detailed data is collected at 5-minute intervals; when stress is low, the interval is adjusted to 30 minutes to reduce user burden. The AI model is trained on a parallel computing cluster using GPUs with supervised learning (e.g., stress-labeled vital and multimodal datasets) and reinforcement learning (e.g., optimization of abnormality detection rate and user burden for each collection frequency). Unlike conventional human-based stress determination or simple fixed collection schedules, the collection unit automatically performs integrated analysis of high-dimensional multimodal data, nonlinear collection timing optimization, and context-dependent control using AI, resulting in significant improvements in important data acquisition efficiency, anomaly detection accuracy, and user burden balance. Technical effects include: (1) improved abnormality detection rate by optimizing collection frequency according to stress state; (2) reduction of storage and communication load by eliminating unnecessary data; (3) reduction of user burden; and so on. Specific application fields include home monitoring for diabetic patients, stress management support, health management for athletes, and health monitoring in nursing care facilities. Furthermore, by linking multiple stress estimation models (e.g., CNN for facial images, RNN for voice, vital data integration model) and collection timing optimization models, more advanced personalized data collection can be realized.

The analysis unit can analyze the user's meal content in real time and predict changes in blood glucose levels after meals. For example, the analysis unit can analyze the ingredients and nutrients consumed by the user and predict changes in blood glucose levels based on this information. The analysis unit can also analyze the amount of carbohydrates and fats in the user's meal content and predict their impact on blood glucose levels. Furthermore, the analysis unit can propose countermeasures to suppress postprandial blood glucose elevation based on the user's meal content. By analyzing the user's meal content in real time, more accurate prediction of changes in blood glucose levels is possible. Specifically, the analysis unit acquires ingredient ID, recipe ID, intake amount, nutrient amounts (e.g., numerical vectors for carbohydrates, fats, proteins, dietary fiber, etc., shape=[5]), meal time, and pre-and post-meal vital data (e.g., time-series tensor shape=[3,10] for blood glucose, heart rate, body temperature) entered by the user at mealtime in real time and inputs them into an AI model. The analysis unit uses convolutional neural networks and Transformer-based multivariate time-series analysis models to automatically learn the complex causal relationships among meal content, nutrients, and vital variation, and predicts blood glucose variation with high accuracy. Examples of AI input include (1) vital data immediately after consuming bread, eggs, and milk for breakfast; (2) blood glucose trends after consuming fish, rice, and vegetables for lunch; (3) blood glucose and heart rate data after consuming a high-fat dinner. Examples of AI model output include “Predicted blood glucose level 1 hour after meal: 160 mg/dL,” “Risk score for rapid blood glucose increase when carbohydrate intake exceeds 50 g: 0.82,” and “Prediction of blood glucose stability with high fat intake.” The analysis unit automatically generates countermeasures to suppress blood glucose elevation (e.g., recommend light exercise within 30 minutes after meals, suggest increased water intake, propose carbohydrate restriction for the next meal) based on these outputs and displays them in real time on the user terminal UI. The AI model is trained on a parallel computing cluster using GPUs with supervised learning (e.g., datasets labeled with meal content, nutrients, vital data, and blood glucose variation) and reinforcement learning (e.g., optimization with blood glucose stability after meal suggestion as reward). Unlike conventional human-based meal record reference or simple rule-based prediction, the analysis unit automatically performs integrated analysis of high-dimensional multivariate data, nonlinear causal inference, and real-time countermeasure generation using AI, resulting in significant improvements in prediction accuracy, speed, and personalization of blood glucose variation. Technical effects include: (1) improved prediction accuracy by considering meal content factors; (2) early detection of rapid blood glucose increase risk; (3) personalized countermeasure suggestions for each user; (4) reduction of burden on medical professionals and family; and so on. Specific application fields include home blood glucose management for diabetic patients, health management support, nutrition management for athletes, and meal management in nursing care facilities. Furthermore, by linking multiple meal analysis models (e.g., nutrient estimation model+blood glucose variation prediction model+countermeasure suggestion generation model), more advanced personalized health support can be realized.

The notification unit can estimate the user's emotion and customize notification content based on the estimated emotion of the user. For example, if the user is feeling stressed, the notification unit can provide simple and easy-to-understand notification content. If the user is relaxed, the notification unit can provide notification content with detailed information. If the user is in a hurry, the notification unit can provide concise notifications focusing on key points. By customizing notification content according to the user's emotion, more appropriate notifications can be provided. Specifically, the notification unit inputs multimodal data such as the user's facial images (e.g., face image tensor shape=[3,224,224]), voice waveforms (e.g., 1D array, sampling rate 16 kHz), and text data (e.g., speech recognition results or user input) into an AI model. The notification unit uses convolutional neural networks to extract feature maps from facial images, and recurrent neural networks or Transformer-based models to estimate emotional states (e.g., stress, relaxation, hurry, etc.) from voice and text. Examples of AI input include (1) facial images before a meeting, (2) voice data when in a hurry, and (3) text such as “I want to know the result immediately.” Examples of AI model output include “Emotion label: stress, score: 0.75,” “Emotion label: relaxation, score: 0.12,” and “Emotion label: hurry, score: 0.65.” The notification unit automatically optimizes the notification content generation logic based on these emotion estimation results. For example, during stress, minimize graphs and numerical data, use color coding and icons for intuitive display; during relaxation, add detailed graphs, statistics, and trend information; when in a hurry, emphasize key points with large fonts. The AI model is trained on a parallel computing cluster using GPUs with supervised learning (e.g., emotion-labeled notification content selection datasets) and reinforcement learning (e.g., optimization of notification content based on user satisfaction as reward). These notification content optimization results are reflected in real time on user terminals and medical professional terminals, enabling optimal information delivery according to the user's condition. Unlike conventional human-based emotion determination or fixed notification content, the notification unit automatically performs integrated analysis of high-dimensional multimodal data, nonlinear notification content optimization, and context-dependent control using AI, resulting in significant improvements in usability, information delivery efficiency, and satisfaction. Technical effects include: (1) optimization of information delivery efficiency according to emotional state; (2) prevention of misunderstanding and oversight; (3) personalized notification for each user; (4) reduction of burden on medical professionals and family; and so on. Specific application fields include home blood glucose management for diabetic patients, stress management support, health monitoring for athletes, and health management in nursing care facilities. Furthermore, by linking multiple emotion estimation models (e.g., CNN for facial images, RNN for voice, Transformer for text) and notification content generation models, more advanced personalized notifications can be realized.

The suggestion unit can analyze the user's exercise history and provide meal suggestions after exercise. For example, the suggestion unit can analyze the user's exercise intensity and duration to propose ingredients and recipes suitable for post-exercise meals. The suggestion unit can also propose meals suitable for energy replenishment after exercise. Furthermore, the suggestion unit can provide meal suggestions considering nutritional balance after exercise based on the user's exercise history. By considering the user's exercise history, more appropriate meal suggestions can be provided. Specifically, the suggestion unit acquires the user's exercise history (e.g., time-series array shape=[5,30] for exercise type, intensity, duration, calories burned, exercise start/end time), post-exercise vital data (e.g., time-series tensors for heart rate, blood glucose level, body temperature), and meal history data (e.g., recent meal content, intake time, nutrient amounts) from a cloud database and inputs them into an AI model. The suggestion unit uses recurrent neural networks or Transformer-based multivariate time-series analysis models to automatically learn the complex causal relationships among exercise history, vital variation, and meal content, and generates optimal meal suggestions after exercise. Examples of AI input include (1) heart rate, blood glucose level, and body temperature data after 30 minutes of running; (2) calories burned and vital data after strength training; (3) meal content and blood glucose trends after walking. Examples of AI model output include “Recommend consuming banana and yogurt within 30 minutes after exercise,” “After high-intensity exercise, suggest high-protein and high-carbohydrate meals,” and “Recommend low-GI foods for stable blood glucose after exercise.” These outputs are displayed in natural language on the user terminal UI, providing personalized meal suggestions in real time according to the user's exercise history. The AI model is trained on a parallel computing cluster using GPUs with supervised learning (e.g., datasets labeled with exercise history, vital data, and meal suggestions) and reinforcement learning (e.g., optimization with blood glucose stability after suggestion as reward). Unlike conventional human-based exercise record reference or simple recipe recommendation, the suggestion unit automatically performs integrated analysis of high-dimensional multivariate data, nonlinear causal inference, and personalized suggestion generation using AI, resulting in significant improvements in meal suggestion accuracy, speed, and user satisfaction. Technical effects include: (1) improved meal suggestion accuracy by considering exercise history factors; (2) suppression of blood glucose variation; (3) improvement of user satisfaction; (4) reduction of burden on medical professionals and family; and so on. Specific application fields include home meal management for diabetic patients, nutrition management for athletes, health management support, and meal suggestions in nursing care facilities. Furthermore, by linking multiple exercise analysis models (e.g., exercise type estimation model+vital variation prediction model+meal suggestion generation model), more advanced personalized meal suggestions can be realized.

The collection unit can estimate the user's emotion and adjust the vital data collection method based on the estimated emotion of the user. For example, if the user is feeling stressed, the collection unit can increase the collection frequency to obtain more detailed data. If the user is relaxed, the collection unit can reduce the collection frequency to lessen the user's burden. If the user is exercising, the collection unit can adjust the post-exercise collection timing to obtain accurate data. By adjusting the vital data collection method according to the user's emotion, more accurate data can be obtained. Specifically, the collection unit inputs the user's facial images (e.g., face image tensor shape=[3,224,224]), voice waveforms (e.g., 1D array, sampling rate 16 kHz), text data (e.g., speech recognition results or user input), and vital data such as heart rate, blood pressure, body temperature, and blood glucose level (e.g., time-series tensor shape=[4,60]) into an AI model. The collection unit uses convolutional neural networks to extract feature maps from facial images, and recurrent neural networks or Transformer-based models to estimate emotional states from voice, text, and vital time-series data. Examples of AI input include (1) facial images and heart rate data during a meeting, (2) voice data and blood pressure data during exercise, (3) text such as “I'm feeling relaxed today.” Examples of AI model output include “Emotion label: stress, score: 0.82,” “Emotion label: relaxation, score: 0.15,” and “Emotion label: exercising, score: 0.65.” The collection unit automatically optimizes the control logic for vital data collection frequency and timing based on these emotion estimation results. For example, during stress, detailed data is collected at 5-minute intervals; during relaxation, the interval is adjusted to 30 minutes; during exercise, the collection unit detects exercise state from accelerometer and heart rate changes and controls collection to occur within 10 minutes after exercise. The AI model is trained on a parallel computing cluster using GPUs with supervised learning (e.g., emotion-labeled vital and collection history datasets) and reinforcement learning (e.g., optimization of abnormality detection rate and user burden for each collection strategy). Unlike conventional human-based emotion determination or simple fixed collection schedules, the collection unit automatically performs integrated analysis of high-dimensional multimodal data, nonlinear collection method optimization, and context-dependent control using AI, resulting in significant improvements in data acquisition efficiency, anomaly detection accuracy, and user burden balance. Technical effects include: (1) improved abnormality detection rate by optimizing collection method according to emotional state; (2) reduction of storage and communication load by eliminating unnecessary data; (3) reduction of user burden; and so on. Specific application fields include home monitoring for diabetic patients, stress management support, health management for athletes, and health monitoring in nursing care facilities. Furthermore, by linking multiple emotion estimation models (e.g., CNN for facial images, RNN for voice, Transformer for text) and collection method optimization models, more advanced personalized data collection can be realized.

The analysis unit can predict changes in blood glucose levels by taking into account the user's geographic location information. For example, if the user is in a high-altitude area, the analysis unit can predict changes in blood glucose levels based on oxygen saturation data. If the user is in an urban area, the analysis unit can predict changes in blood glucose levels based on heart rate and blood pressure data. If the user is at home, the analysis unit can predict changes in blood glucose levels based on body temperature and blood glucose level data. By considering the user's geographic location information, changes in blood glucose levels can be predicted more accurately. Specifically, the analysis unit acquires location information (e.g., latitude, longitude, altitude, location accuracy, timestamp, etc. as structured data) in real time from GPS sensors or Wi-Fi/Bluetooth beacons, and integrates it with vital data (time-series tensors of heart rate, blood pressure, body temperature, and blood glucose levels) as input to an AI model. The analysis unit uses a Transformer-based spatiotemporal multivariate analysis model to automatically learn the correlation and environmental dependencies between location information and vital data, and predicts blood glucose variation with high accuracy. Examples of AI input include (1) vital data at high altitude (elevation 2000 m), (2) data in urban areas (with latitude/longitude, elevation, temperature, PM2.5 concentration, etc.), and (3) data at home (determined by Wi-Fi beacon ID). Examples of AI model output include: “Current location: high altitude, predicted blood glucose elevation risk due to decreased oxygen saturation: 0.68”; “Current location: urban area, predicted blood glucose elevation due to heart rate and blood pressure variation: 170 mg/dL”; “Current location: home, predicted stable blood glucose and body temperature: 120 mg/dL.” These outputs are passed to subsequent notification units or insulin administration control units and used for threshold determination and branching processing (e.g., emergency notification when risk increases at high altitude, regular notification in normal cases). The AI model is trained on a parallel computing cluster using GPUs with supervised learning (e.g., location-labeled vital and blood glucose datasets) and reinforcement learning (e.g., optimization of blood glucose variation for each location). Unlike conventional human-based location determination or simple mass data collection, the analysis unit automatically performs multivariate analysis of high-dimensional spatiotemporal data, nonlinear priority inference, and rule-based prediction control using AI, resulting in significant improvements in relevant data acquisition, anomaly detection accuracy, and operational efficiency. Technical effects include: (1) early detection of health risks with high location dependency; (2) reduction of storage and communication load by eliminating unnecessary data; (3) reduction of user burden; and so on. Specific application fields include health management for mountaineers and high-altitude residents, lifestyle disease monitoring in urban areas, home medical support, and health management in nursing care facilities. Furthermore, by linking multiple AI models (e.g., location estimation model+vital priority estimation model), more advanced personalized prediction can be realized.

The notification unit can estimate the user's emotion and determine the priority of notifications based on the estimated emotion of the user. For example, if the user is feeling stressed, the notification unit can prioritize only important notifications. If the user is relaxed, the notification unit can prioritize detailed notifications. If the user is exercising, the notification unit can prioritize important notifications after exercise. By determining the priority of notifications according to the user's emotion, important notifications can be prioritized. Specifically, the notification unit inputs multimodal data such as the user's facial images (e.g., face image tensor shape=[3,224,224]), voice waveforms (e.g., 1D array, sampling rate 16 kHz), and text data (e.g., speech recognition results or user input) into an AI model. The notification unit uses convolutional neural networks to extract feature maps from facial images, and recurrent neural networks or Transformer-based models to estimate emotional states (e.g., stress, relaxation, exercising, etc.) from voice and text. Examples of AI input include (1) facial images during a meeting, (2) voice data during exercise, and (3) app input text. Examples of AI model output include “Emotion label: stress, score: 0.82,” “Emotion label: relaxation, score: 0.15,” and “Emotion label: exercising, score: 0.65.” The notification unit automatically determines the notification priority control logic (e.g., emergency notifications only during stress, detailed notifications during relaxation, important notifications prioritized after exercise) based on these emotion estimation results. The AI model is trained on a parallel computing cluster using GPUs with supervised learning (e.g., emotion-labeled notification history datasets) and reinforcement learning (e.g., optimization of user satisfaction for each notification priority). Unlike conventional human-based emotion determination or simple mass notification, the notification unit automatically performs integrated analysis of high-dimensional multimodal data, nonlinear priority inference, and rule-based notification control using AI, resulting in significant improvements in important notification acquisition efficiency, anomaly detection accuracy, and user burden balance. Technical effects include: (1) improved anomaly detection rate by high-accuracy notification during important events; (2) reduction of storage and communication load by eliminating unnecessary notifications; (3) reduction of user burden; and so on. Application fields include home monitoring for diabetic patients, stress management support, condition management for athletes, and health monitoring in nursing care settings. Furthermore, by combining multiple emotion estimation models (e.g., CNN for facial images, RNN for voice, Transformer for text), notification priority estimation accuracy and notification control optimization can be further enhanced.

The following is a brief description of the processing flow of Example of the Embodiment. Specifically, the system comprises multiple functional modules such as a collection unit, analysis unit, notification unit, and suggestion unit, which cooperate to acquire, analyze, notify, and suggest various types of data in real time, including the user's vital data, emotion, exercise history, meal history, geographic location information, sleep patterns, and social media activity. Each module uses AI architectures such as convolutional neural networks, recurrent neural networks, and Transformer-based models to automatically perform integrated analysis of multimodal data, nonlinear parameter optimization, and context-dependent control. The AI model applies various learning methods such as supervised learning, reinforcement learning, transfer learning, and self-supervised learning on a parallel computing cluster using GPUs to realize personalized health support for each user. As a result, unlike conventional human-based simple data reference or rule-based control, the overall system achieves significant improvements in accuracy, speed, reproducibility, and scalability. Technical effects include: (1) high-accuracy data acquisition and improved anomaly detection rate during important events; (2) reduction of storage and communication load by eliminating unnecessary data, notifications, and suggestions; (3) reduction of burden on users, medical professionals, and family; (4) improved adaptability to diverse use cases; and so on. Specific application fields include home blood glucose management for diabetic patients, remote medical support, health monitoring in nursing care facilities, health management for athletes, health management support, stress management, nutrition guidance, rehabilitation support, and lifestyle disease prevention.

Step 1: The collection unit collects vital data. Vital data includes heart rate, blood pressure, body temperature, blood glucose levels, and the like. The collection unit can collect vital data obtained from wearable devices in real time. Furthermore, the collection unit can adjust the timing of vital data collection using AI. For example, the collection unit can estimate the user's emotion and adjust the timing of vital data collection based on the estimated emotion of the user. Step 2: The analysis unit analyzes the data collected by the collection unit and predicts changes in blood glucose levels. The analysis unit can use AI to predict increases or decreases in blood glucose levels based on past and current data. For example, it can predict increases in blood glucose levels after meals or decreases after exercise. Step 3: The notification unit notifies the timing of insulin injection based on the predicted changes in blood glucose levels by the analysis unit. The notification unit can use AI to notify the timing for insulin injection before blood glucose levels rise. Step 4: The notification unit notifies the user and contacts when hypoglycemia is detected. The notification unit can use AI to detect hypoglycemia and notify not only the user but also registered contacts in a timely manner. Specifically, in Step 1, the system acquires multidimensional vital data such as heart rate, blood pressure, body temperature, and blood glucose levels (e.g., a time-series tensor with shape=[4,60] for every minute) in real time from wearable devices or smartphones, and inputs them into the AI model of the collection unit (e.g., CNN for facial expressions, RNN for voice, and a vital data integration model). The collection unit estimates the user's emotional state from facial images, voice waveforms, text data, etc., and automatically optimizes the collection timing according to the emotional state, such as every 5 minutes during stress and every 30 minutes during relaxation. In Step 2, the analysis unit integrates multivariate data such as collected vital data, meal history, exercise history, sleep patterns, and geographic location information, and uses a Transformer-based time-series analysis model or a multimodal integration model to predict blood glucose level fluctuations with high accuracy. Examples of AI input include (1) vital data after meals, (2) heart rate and blood glucose data after exercise, (3) body temperature and blood glucose data during sleep deprivation, and (4) vital data at high altitudes. Examples of AI model output include “Predicted blood glucose level after 1 hour: 180 mg/dL” and “Hypoglycemia risk score: 0.85”. In Step 3, the notification unit dynamically controls the insulin injection timing notification logic based on the output results of the analysis unit and provides optimal notifications in real time to user terminals and medical professional terminals. The AI model is trained on a parallel computing cluster using GPUs through supervised learning (e.g., datasets of vital data, emotion, meal, exercise, sleep, location, blood glucose level fluctuations, notification history) and reinforcement learning (e.g., optimization of blood glucose stability and user satisfaction for each notification timing). In Step 4, when the hypoglycemia risk score exceeds a threshold, the notification unit automatically sends emergency notifications to user terminals and registered contacts (e.g., family members, medical professionals' smartphones, smartwatches, etc.). Unlike conventional human data referencing or simple fixed schedule notifications, this system automatically performs integration analysis of high-dimensional multimodal data, nonlinear parameter optimization, and context-dependent control using AI, thereby greatly improving the accuracy, speed, and usability of blood glucose level fluctuation prediction, notification timing, and anomaly detection. Technical effects include (1) high-accuracy data acquisition and improved anomaly detection rate during important events, (2) reduction of storage and communication load by eliminating unnecessary data and notifications, (3) reduced burden on users, medical professionals, and family members, and (4) improved adaptability to diverse use cases. Specific application fields include home blood glucose management for diabetic patients, remote medical support, health monitoring in nursing facilities, health management for athletes, health management support, stress management, nutritional guidance, rehabilitation support, and prevention of lifestyle-related diseases.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned collection unit, analysis unit, notification unit, and suggestion unit is implemented by at least one of, for example, the smart device 14 and the data processing apparatus 12. For example, the collection unit collects vital data using sensors of the smart device 14 and transmits it to the data processing apparatus 12 via the control unit 46A. The analysis unit is implemented, for example, by a specific processing unit 290 of the data processing apparatus 12, and analyzes the collected vital data to predict changes in blood glucose levels. The notification unit is implemented, for example, by the control unit 46A of the smart device 14, and performs notification of the timing of insulin injection and hypoglycemia. The suggestion unit is implemented, for example, by the specific processing unit 290 of the data processing apparatus 12, and provides meal suggestions to the user. The correspondence between each unit and the apparatus or control unit is not limited to the examples described above and various modifications are possible.

### Second Embodiment

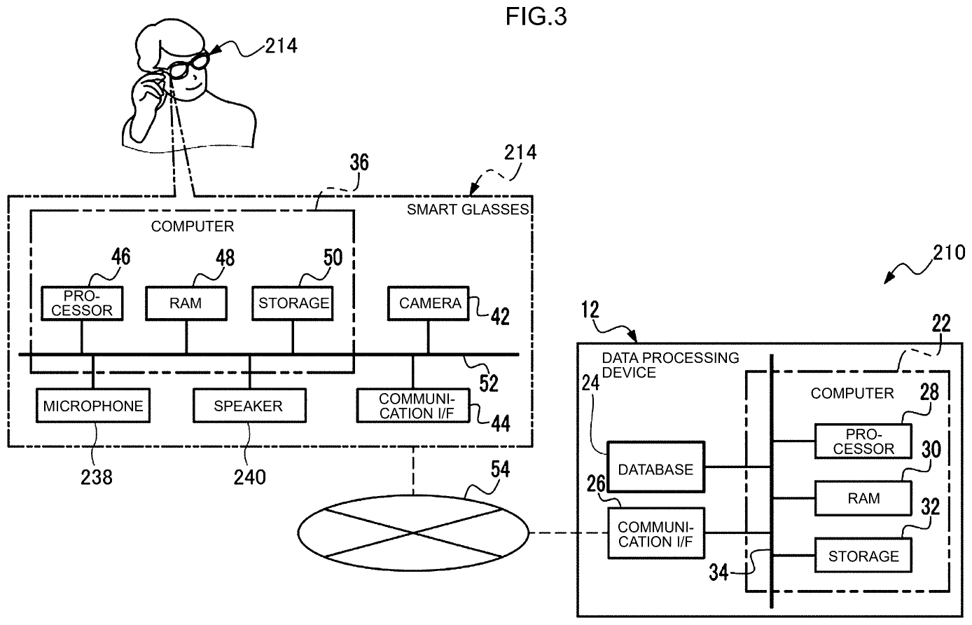

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

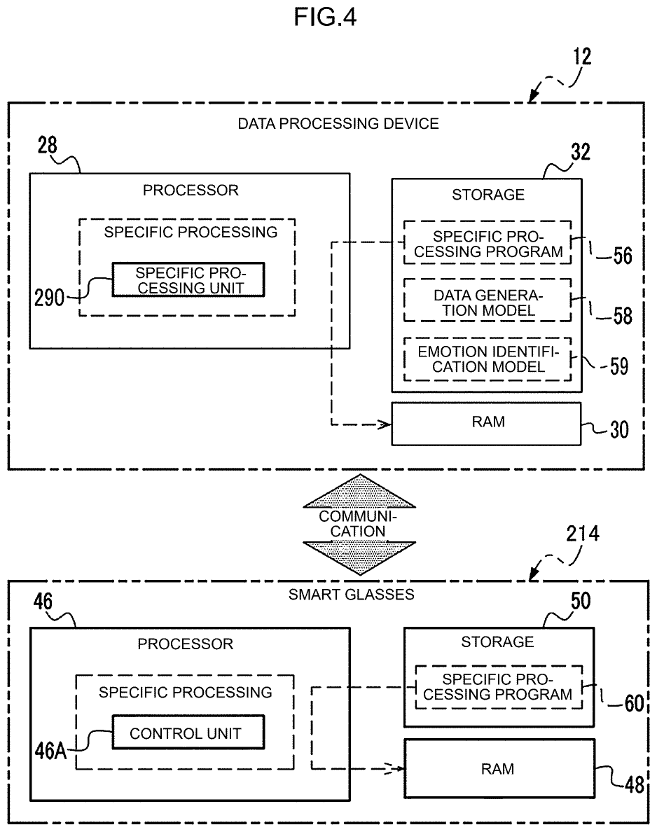

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned collection unit, analysis unit, notification unit, and suggestion unit is implemented by at least one of, for example, the smart glasses 214 and the data processing apparatus 12. For example, the collection unit collects vital data using sensors of the smart glasses 214 and transmits it to the data processing apparatus 12 via the control unit 46A. The analysis unit is implemented, for example, by a specific processing unit 290 of the data processing apparatus 12, and analyzes the collected vital data to predict changes in blood glucose levels. The notification unit is implemented, for example, by the control unit 46A of the smart glasses 214, and performs notification of the timing of insulin injection and hypoglycemia. The suggestion unit is implemented, for example, by the specific processing unit 290 of the data processing apparatus 12, and provides meal suggestions to the user. The correspondence between each unit and the apparatus or control unit is not limited to the examples described above and various modifications are possible.

### Third Embodiment

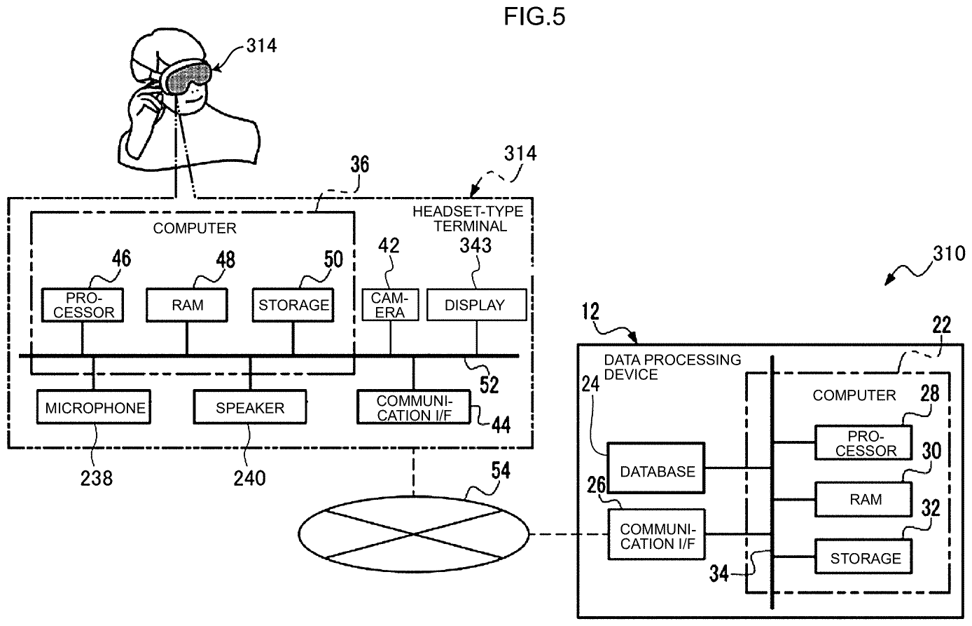

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

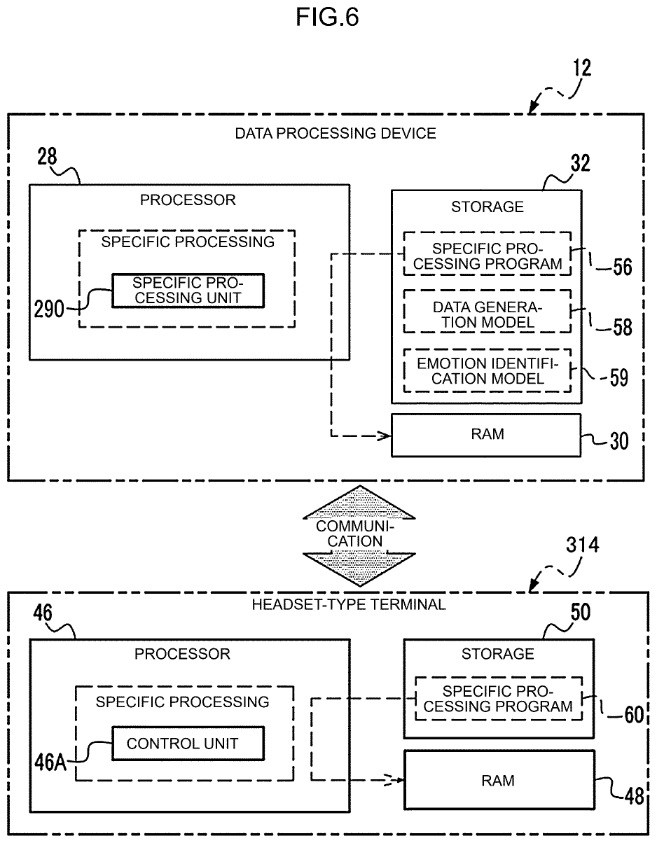

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned collection unit, analysis unit, notification unit, and suggestion unit is implemented by at least one of, for example, the headset-type terminal 314 and the data processing apparatus 12. For example, the collection unit collects vital data using sensors of the headset-type terminal 314 and transmits it to the data processing apparatus 12 via the control unit 46A. The analysis unit is implemented, for example, by a specific processing unit 290 of the data processing apparatus 12, and analyzes the collected vital data to predict changes in blood glucose levels. The notification unit is implemented, for example, by the control unit 46A of the headset-type terminal 314, and performs notification of the timing of insulin injection and hypoglycemia. The suggestion unit is implemented, for example, by the specific processing unit 290 of the data processing apparatus 12, and provides meal suggestions to the user. The correspondence between each unit and the apparatus or control unit is not limited to the examples described above and various modifications are possible.

### Fourth Embodiment

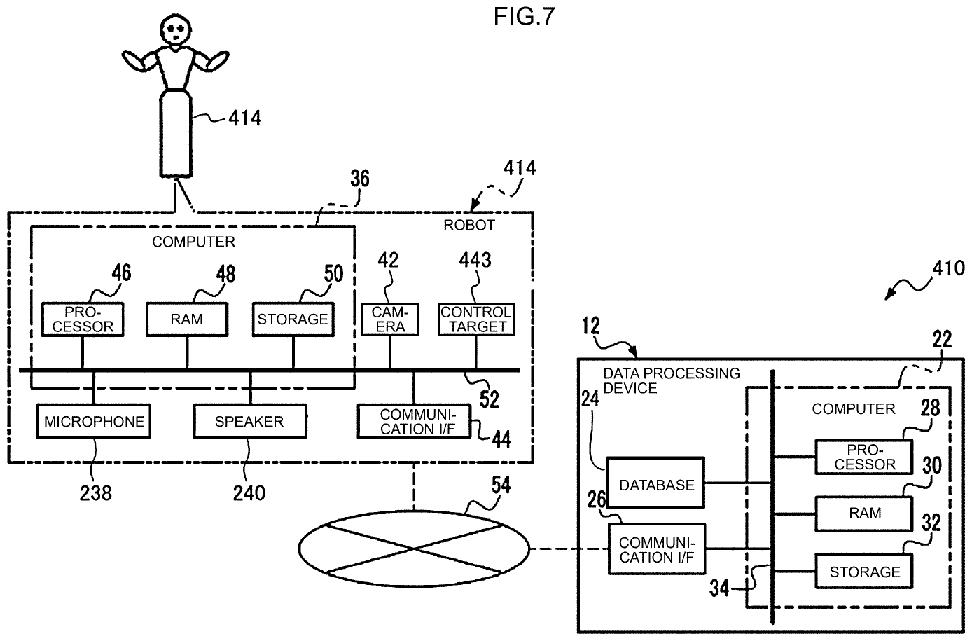

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

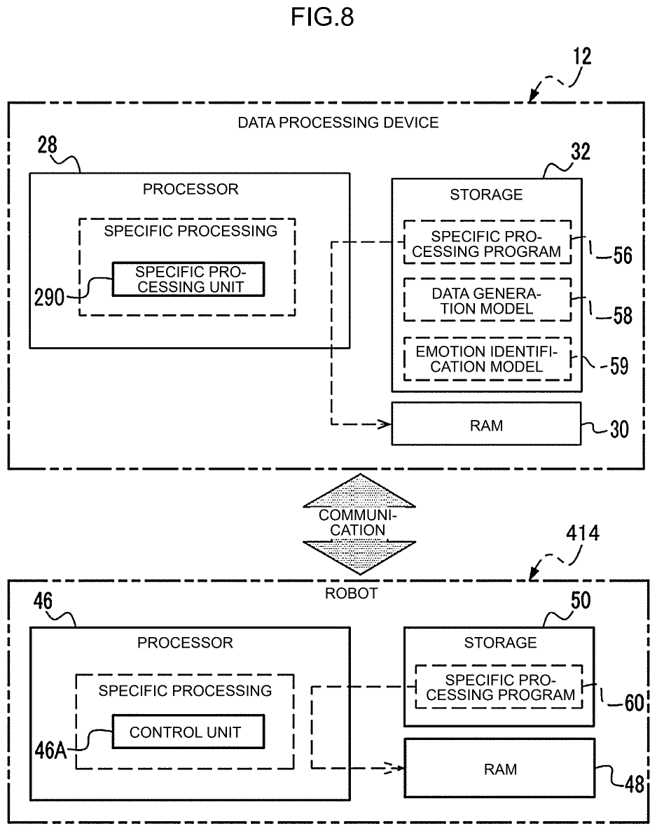

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned collection unit, analysis unit, notification unit, and suggestion unit is implemented by at least one of, for example, the robot 414 and the data processing apparatus 12. For example, the collection unit collects vital data using sensors of the robot 414 and transmits it to the data processing apparatus 12 via the control unit 46A. The analysis unit is implemented, for example, by a specific processing unit 290 of the data processing apparatus 12, and analyzes the collected vital data to predict changes in blood glucose levels. The notification unit is implemented, for example, by the control unit 46A of the robot 414, and performs notification of the timing of insulin injection and hypoglycemia. The suggestion unit is implemented, for example, by the specific processing unit 290 of the data processing apparatus 12, and provides meal suggestions to the user. The correspondence between each unit and the apparatus or control unit is not limited to the examples described above and various modifications are possible.

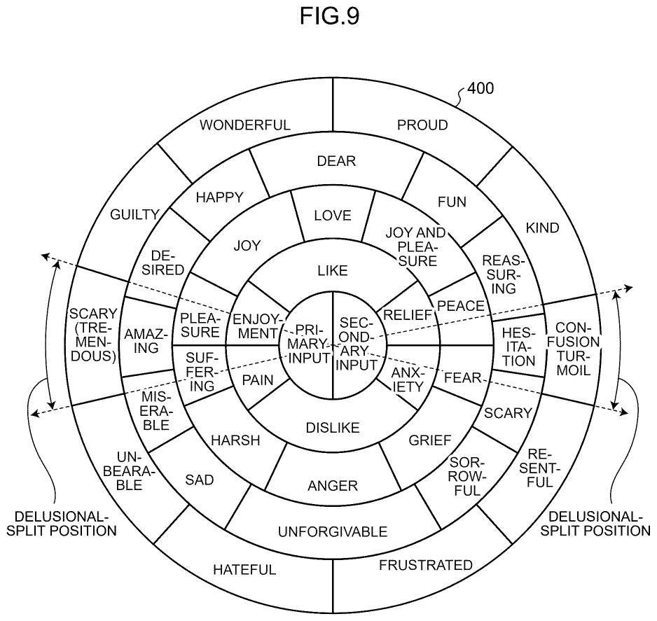

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

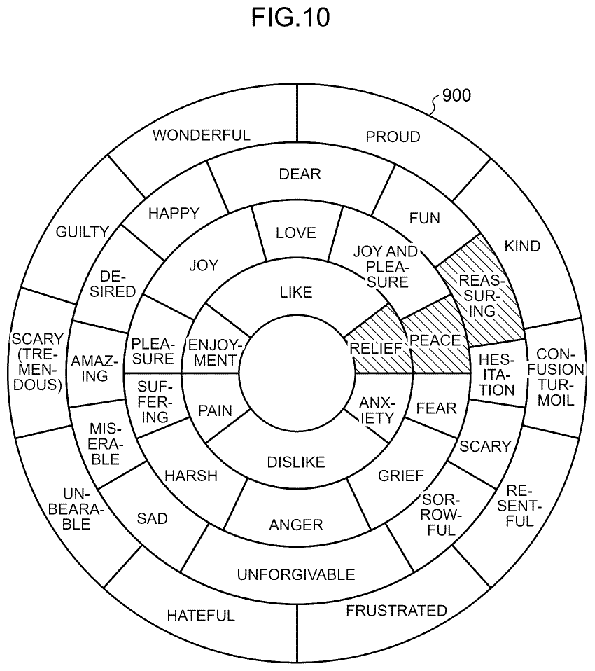

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

### Supplementary Note 1

A system comprising: a collection unit configured to collect vital data; an analysis unit configured to analyze data collected by the collection unit and predict changes in blood glucose levels; a notification unit configured to notify the timing of insulin injection based on the predicted changes in blood glucose levels by the analysis unit; and a notification unit configured to notify the user and contacts when hypoglycemia is detected.

### Supplementary Note 2

The system according to Supplementary Note 1, further comprising a suggestion unit configured to provide meal suggestions.

### Supplementary Note 3

The system according to Supplementary Note 1, wherein the collection unit is configured to estimate the user's emotion and adjust the timing of vital data collection based on the estimated emotion of the user.

### Supplementary Note 4

The system according to Supplementary Note 1, wherein the collection unit is configured to analyze the user's past vital data collection history and select an appropriate collection method.

### Supplementary Note 5

The system according to Supplementary Note 1, wherein the collection unit is configured to perform filtering based on the user's current activity status or environment when collecting vital data.

### Supplementary Note 6

The system according to Supplementary Note 1, wherein the collection unit is configured to estimate the user's emotion and determine the priority of vital data to be collected based on the estimated emotion of the user.

### Supplementary Note 7

The system according to Supplementary Note 1, wherein the collection unit is configured to preferentially collect highly relevant data based on the user's geographic location information when collecting vital data.

### Supplementary Note 8

The system according to Supplementary Note 1, wherein the collection unit is configured to analyze the user's social media activity and collect relevant data when collecting vital data.

### Supplementary Note 9

The system according to Supplementary Note 1, wherein the analysis unit is configured to estimate the user's emotion and adjust the accuracy of blood glucose level change prediction based on the estimated emotion of the user.

### Supplementary Note 10

The system according to Supplementary Note 1, wherein the analysis unit is configured to improve prediction accuracy by comparing past vital data with current vital data during analysis.

### Supplementary Note 11

The system according to Supplementary Note 1, wherein the analysis unit is configured to predict changes in blood glucose levels based on the user's lifestyle or meal history during analysis.

### Supplementary Note 12

The system according to Supplementary Note 1, wherein the analysis unit is configured to estimate the user's emotion and adjust a display method of analysis results based on the estimated emotion of the user.

### Supplementary Note 13

The system according to Supplementary Note 1, wherein the analysis unit is configured to predict changes in blood glucose levels by taking into account the user's geographic location information during analysis.

### Supplementary Note 14

The system according to Supplementary Note 1, wherein the analysis unit is configured to analyze the user's social media activity and predict changes in blood glucose levels using relevant data during analysis.

### Supplementary Note 15

The system according to Supplementary Note 1, wherein the notification unit is configured to estimate the user's emotion and adjust the timing of notification based on the estimated emotion of the user.

### Supplementary Note 16

The system according to Supplementary Note 1, wherein the notification unit is configured to refer to the user's past response history at the time of notification and select an optimal notification method.

### Supplementary Note 17

The system according to Supplementary Note 1, wherein the notification unit is configured to customize notification content based on the user's current activity status or environment at the time of notification.

### Supplementary Note 18

The system according to Supplementary Note 1, wherein the notification unit is configured to estimate the user's emotion and determine the priority of notification based on the estimated emotion of the user.

### Supplementary Note 19

The system according to Supplementary Note 1, wherein the notification unit is configured to select an optimal notification method by taking into account the user's geographic location information at the time of notification.

### Supplementary Note 20

The system according to Supplementary Note 1, wherein the notification unit is configured to analyze the user's social media activity at the time of notification and notify relevant information.

### Supplementary Note 21

The system according to Supplementary Note 2, wherein the suggestion unit is configured to estimate the user's emotion and adjust a meal suggestion method based on the estimated emotion of the user.

### Supplementary Note 22

The system according to Supplementary Note 2, wherein the suggestion unit is configured to refer to the user's past meal history at the time of suggestion and provide an optimal suggestion.

### Supplementary Note 23

The system according to Supplementary Note 2, wherein the suggestion unit is configured to customize meal suggestions based on the user's current health status or lifestyle at the time of suggestion.

### Supplementary Note 24

The system according to Supplementary Note 2, wherein the suggestion unit is configured to estimate the user's emotion and determine the priority of meal suggestions based on the estimated emotion of the user.

### Supplementary Note 25

The system according to Supplementary Note 2, wherein the suggestion unit is configured to provide optimal meal suggestions by taking into account the user's geographic location information at the time of suggestion.

### Supplementary Note 26

The system according to Supplementary Note 2, wherein the suggestion unit is configured to analyze the user's social media activity at the time of suggestion and provide relevant meal suggestions.

What is claimed is:

1. A system comprising:
circuitry configured to:
acquire, from a sensor device communicatively coupled to the system via a packet-switched network, time-series sensor data representing a multidimensional tensor of sequential measurements;
generate, by inputting the time-series sensor data into a trained neural network comprising a recurrent neural network or a Transformer-based time-series analysis model, a prediction value indicating a future state change derived from the time-series sensor data;
generate, by comparing the prediction value against a threshold, a notification signal indicating a recommended action associated with the future state change; and
transmit, to a client terminal communicatively coupled to the system via the packet-switched network, output data generated based on the notification signal.

2. The system according to claim 1, wherein the time-series sensor data comprises a plurality of sensor channels including at least two of a cardiac rhythm channel, a thermal channel, a pressure channel, or a biochemical level channel, and the multidimensional tensor has a shape defined by a number of the plurality of sensor channels and a number of temporal samples.

3. The system according to claim 1, wherein the circuitry is further configured to generate, by inputting the time-series sensor data into a data generation model comprising a large language model, a natural-language recommendation based on the prediction value, and to include the natural-language recommendation in the output data.

4. The system according to claim 1, wherein the circuitry is further configured to generate, by inputting at least one of a facial image, audio data, or text data from the client terminal into an emotion identification model, an emotion value indicating an estimated emotion of a user of the client terminal, and to adjust a parameter of the trained neural network based on the emotion value.

5. The system according to claim 4, wherein the emotion identification model comprises a multimodal neural network configured to receive the facial image, the audio data, and biometric sensor data, and to output emotion labels as a probability distribution comprising a score of 0.0 to 1.0 for each of a plurality of emotion categories.

6. The system according to claim 4, wherein the circuitry is further configured to adjust a collection frequency of the time-series sensor data based on the emotion value, such that when the emotion value indicates a high-stress state the collection frequency is increased and when the emotion value indicates a low-stress state the collection frequency is decreased.

7. The system according to claim 1, wherein the circuitry is further configured to compare past time-series sensor data stored in a database with current time-series sensor data, and to extract feature differences comprising at least one of a moving average difference, a standard deviation difference, or a peak value difference, and to input the feature differences into the trained neural network to improve accuracy of the prediction value.

8. The system according to claim 1, wherein the circuitry is further configured to acquire lifestyle history data comprising at least one of meal history data, exercise history data, or sleep history data associated with a user of the client terminal, and to input the lifestyle history data together with the time-series sensor data into the trained neural network to generate the prediction value.

9. The system according to claim 8, wherein the meal history data comprises a time-series array of ingredient identifiers, recipe identifiers, intake timestamps, and nutrient amounts, and the trained neural network comprises a Transformer-based multivariate time-series analysis model configured to learn causal relationships among the meal history data and the time-series sensor data.

10. The system according to claim 8, wherein the exercise history data comprises exercise type, intensity, duration, and calories burned, and the circuitry is further configured to selectively apply a sub-model trained on post-exercise sensor data patterns when the exercise history data indicates a recent exercise event.

11. The system according to claim 1, wherein the circuitry is further configured to acquire activity state data from an accelerometer and a heart rate sensor of the sensor device, to classify an activity state of a user as one of resting, exercising, or sleeping by inputting the activity state data into a time-series classification model, and to apply a filtering rule to the time-series sensor data based on the classified activity state.

12. The system according to claim 1, wherein the circuitry is further configured to acquire geographic location data from a positioning sensor of the sensor device, and to input the geographic location data together with the time-series sensor data into a spatiotemporal multivariate analysis model to generate the prediction value with location-dependent weighting.

13. The system according to claim 1, wherein the circuitry is further configured to analyze a past notification response history of a user of the client terminal, the past notification response history comprising notification timestamps, notification methods, and user response flags, and to select an optimal notification method from a plurality of notification methods based on the past notification response history.

14. The system according to claim 1, wherein the circuitry is further configured to acquire environment data comprising at least one of ambient temperature, humidity, illuminance, or atmospheric pressure from an environmental sensor, and to adjust a parameter of the trained neural network based on the environment data.

15. The system according to claim 1, wherein the notification signal comprises a first notification signal transmitted to the client terminal associated with a user and a second notification signal transmitted to a registered contact terminal communicatively coupled to the system via the packet-switched network, and the second notification signal is generated when the prediction value exceeds an emergency threshold.

16. The system according to claim 15, wherein the second notification signal is transmitted via at least one of a short message service, an automated voice call, or a push notification, and the circuitry is further configured to initiate an external service linkage via an application programming interface when the prediction value exceeds the emergency threshold.

17. The system according to claim 1, wherein the circuitry is further configured to generate, by inputting the prediction value, the time-series sensor data, and an emotion value into a data generation model, a content recommendation comprising a natural-language text identifying at least one of suggested items or suggested procedures associated with the prediction value, and to transmit the content recommendation to the client terminal.

18. A system comprising:
circuitry configured to:
acquire, from a sensor device communicatively coupled to the system via a packet-switched network, time-series sensor data representing a multidimensional tensor having a shape defined by a number of sensor channels and a number of temporal samples, the time-series sensor data sampled at intervals of 10 seconds to 1 minute;
generate, by inputting the time-series sensor data for a preceding 24-hour period into a trained neural network comprising at least one of a convolutional neural network, a recurrent neural network, or a Transformer-based time-series analysis model, a prediction value comprising a regression output indicating a future state change and an anomaly score;
generate, by inputting at least one of a facial image, audio data, or text data into an emotion identification model, an emotion value indicating an estimated emotion of a user of the sensor device;
adjust a loss function or a smoothing parameter of the trained neural network based on the emotion value, such that when the emotion value indicates stress a loss function emphasizing nonlinear changes is applied and when the emotion value indicates relaxation a smoothing parameter is strengthened;
generate, by comparing the prediction value against a threshold, a notification signal indicating a recommended action; and
transmit, to a client terminal communicatively coupled to the system via the packet-switched network, output data generated based on the notification signal, and transmit a second notification signal to a registered contact terminal when the anomaly score exceeds an emergency threshold.

19. The system according to claim 18, wherein the circuitry is further configured to input the prediction value, the time-series sensor data, and the emotion value into a data generation model comprising a large language model, and to generate a natural-language content recommendation identifying suggested items that suppress the future state change, the natural-language content recommendation adjusted in tone based on the emotion value.

20. A method performed by circuitry of a system, the method comprising:
acquiring, from a sensor device communicatively coupled to the system via a packet-switched network, time-series sensor data representing a multidimensional tensor of sequential measurements;
generating, by inputting the time-series sensor data into a trained neural network comprising a recurrent neural network or a Transformer-based time-series analysis model, a prediction value indicating a future state change derived from the time-series sensor data;
generating, by comparing the prediction value against a threshold, a notification signal indicating a recommended action associated with the future state change; and
transmitting, to a client terminal communicatively coupled to the system via the packet-switched network, output data generated based on the notification signal.
